(19) United States
(12) Patent Application Publication
(10) Pub. No.: US 2007/0154989 A1
(43) Pub. Date: Jul. 5, 2007

(54) ZINC FINGER DOMAINS SPECIFICALLY BINDING AGC

(75) Inventor: Carlos F. Barbas III, Solana Beach, CA (US)

Correspondence Address: CATALYST LAW GROUP, APC, 9710 SCRANTON ROAD, SUITE S-170, SAN DIEGO, CA, 92121, US

(73) Assignee: The Scripps Research Institute, La Jolla, CA (US)

(21) Appl. No.: 11/613,075

(22) Filed: Dec. 19, 2006

Related U.S. Application Data
(60) Provisional application No. 60/756,083, filed on Jan. 3, 2006.

(51) Int. Cl. C07H 21/04; C12P 21/06; C12N 9/22; C12N 15/09

(52) U.S. Cl. 435/69.1; 435/199; 435/320.1; 435/325; 536/23.5; 536/23.2

(57) ABSTRACT

Polypeptides that contain zinc finger-nucleotide binding regions that bind to nucleotide sequences of the formula AGC are provided. Compositions containing a plurality of polypeptides, isolated heptapeptides possessing specific binding activity, polynucleotides that encode such polypeptides and methods of regulating gene expression with such polypeptides, compositions and polynucleotides are also provided.

### CROSS-REFERENCES

This application claims priority from Provisional Application Ser. No. 60/756,083, by Carlos F. Barbas III, entitled “Zinc Finger Domains Specifically Binding AGC” and filed on Jan. 3, 2006, which is incorporated herein in its entirety by this reference.

### GOVERNMENT INTERESTS

Funds used to support some of the studies reported herein were provided by the National Institutes of Health (NIH GM 53910). The United States Government, therefore, may have certain rights in the invention.

### TECHNICAL FIELD OF THE INVENTION

The field of this invention is zinc finger protein binding to target nucleotides. More particularly, the present invention pertains to amino acid residue sequences within the α-helical domain of zinc fingers that specifically bind to target nucleotides of the formula 5′-(AGC)-3′.

### BACKGROUND OF THE INVENTION

The construction of artificial transcription factors has been of great interest in the past years. Gene expression can be specifically regulated by polydactyl zinc finger proteins fused to regulatory domains. Zinc finger domains of the Cys₂-His₂ family have been most promising for the construction of artificial transcription factors due to their modular structure. Each domain consists of approximately 30 amino acids and folds into an α-helical structure stabilized by hydrophobic interactions and chelation of a zinc ion by the conserved Cys₂-His₂ residues. To date, the best characterized protein of this family of zinc finger proteins is the mouse transcription factor Zif 268 [Pavletich et al., (1991) Science 252(5007), 809-817; Elrod-Erickson et al., (1996) Structure 4(10), 1171-1180]. The analysis of the Zif 268/DNA complex suggested that DNA binding is predominantly achieved by the interaction of amino acid residues of the α-helix in position −1, 3, and 6 with the 3′, middle, and 5′ nucleotide of a 3 bp DNA subsite, respectively. Positions 1, 2 and 5 have been shown to make direct or water-mediated contacts with the phosphate backbone of the DNA. Leucine is usually found in position 4 and packs into the hydrophobic core of the domain. Position 2 of the α-helix has been shown to interact with other helix residues and, in addition, can make contact to a nucleotide outside the 3 bp subsite [Pavletich et al., (1991) Science 252(5007), 809-817; Elrod-Erickson etal., (1996) Structure 4(10), 1171-1180; Isalan, M. etal., (1997) Proc Natl Acad Sci USA 94(11), 5617-5621].

The selection of modular zinc finger domains recognizing each of the 5′-(GNN)-3′ DNA subsites with high specificity and affinity and their refinement by site-directed mutagenesis has been demonstrated (U.S. Pat. No. 6,140,081, the disclosure of which is incorporated herein by reference). These modular domains can be assembled into zinc finger proteins recognizing extended 18 bp DNA sequences which are unique within the human genome or any other genome. In addition, these proteins function as transcription factors and are capable of altering gene expression when fused to regulatory domains and can even be made hormone-dependent by fusion to ligand-binding domains of nuclear hormone receptors. To allow the rapid construction of zinc finger-based transcription factors binding to any DNA sequence it is important to extend the existing set of modular zinc finger domains to recognize each of the 64 possible DNA triplets which are assigned meaning in the genetic code. This aim can be achieved by phage display selection and/or rational design. Due to the limited structural data on zinc finger/DNA interaction, rational design of zinc proteins is very time-consuming and may not be possible in many instances. In addition, most naturally occurring zinc finger proteins consist of domains recognizing the 5′-(GNN)-3′ type of DNA sequences. The most promising approach to identify novel zinc finger domains binding to DNA target sequences of the type 5′-(NNN)-3′ is selection via phage display. The limiting step for this approach is the construction of libraries that allow the specification of a 5′ adenine, cytosine or thymine in the subsite recognized by each module. Phage display selections have been based on Zif268 in which different fingers of this protein were randomized [Choo et al., (1994) Proc. Nati. Acad. Sci. U.S. A. 91(23), 11168-72; Rebar et al., (1994) Science (Washington, D.C., 1883-) 263(5147), 671-3; Jamieson et al., (1994) Biochemistry 33, 5689-5695; Wu et al., (1995) PNAS 92, 344-348; Jamieson et al., (1996) Proc Natl Acad Sci USA 93, 12834-12839; Greisman et al., (1997) Science 275(5300), 657-661]. A set of 16 domains recognizing the 5′-(GNN)-3′ type of DNA sequences has previously been reported from a library where finger 2 of C7, a derivative of Zif268 [Wu et al., (1995) PNAS 92, 344-348 Wu, 1995], was randomized [Segal et al., (1999) Proc Natl Acad Sci USA 96(6), 2758-2763]. In such a strategy, selection is limited to domains recognizing 5′-(GNN)-3′ or 5′-(TNN)-3′ due to the Asp² of finger 3 making contact with the complementary base of a 5′ guanine or thymine in the finger-2 subsite [Pavietich et al., (1991) Science 252(5007), 809-817; Elrod-Erickson et al., (1996) Structure 4(10), 1171-1180].

Despite the possible selection of zinc finger domains recognizing sequences of the form 5′-(AGC)-3′ by the strategy described above, in practice such domains having the desired affinity and specificity for this nucleotide triplet have not been obtained. Therefore, there is a need to discover zinc finger domains recognizing sequences of the form 5′-(AGC)-3′ so that a broader “vocabulary” of zinc finger domains is available for the construction of multifinger zinc finger proteins.

The present approach is based on the modularity of zinc finger domains that allows the rapid construction of zinc finger proteins by the scientific community and demonstrates that the concerns regarding limitation imposed by cross-subsite interactions only occurs in a limited number of cases. The present disclosure introduces a new strategy for selection of zinc finger domains specifically recognizing the 5′-(AGC)-3′ type of DNA sequences. Specific DNA-binding properties of these domains were evaluated by a multi-target ELISA against all sixteen 5′-(ANN)-3′ triplets to ensure specificity for 5′-(AGC)-3′. These domains can be readily incorporated into polydactyl proteins containing various numbers of 5′-(AGC)-3′ domains, each specifically recognizing extended 18 bp sequences. Furthermore, these domains can specifically alter gene expression when fused to regulatory domains. These results underline the feasibility of constructing polydactyl proteins from predefined building blocks. In addition, the domains characterized here greatly increase the number of DNA sequences that can be targeted with artificial transcription factors.

### BRIEF SUMMARY OF THE INVENTION

In one aspect, the present invention provides an isolated and purified zinc finger nucleotide binding polypeptide that contains a nucleotide binding region of from 5 to 10 amino acid residues, which region binds preferentially to a target nucleotide of the formula AGC. In one embodiment, a polypeptide of the invention contains a binding region that has an amino acid sequence with the same nucleotide binding characteristics as any of SEQ ID NO: 1 through SEQ ID NO: 57. Such a polypeptide competes for binding to a nucleotide target with any of SEQ ID NO: 1 through SEQ ID NO: 57. That is, a preferred polypeptide contains a binding region that will displace, in a competitive manner, the binding of any of SEQ ID NO: 1 through SEQ ID NO: 57. Means for determining competitive binding are well known in the art. Preferably, the binding region has the amino acid sequence of any of SEQ ID NO: 1 through SEQ ID NO: 57. More preferably, the binding region has the amino acid sequence of any of SEQ ID NO: 1 through SEQ ID NO: 10. Still more preferably, the binding region has the amino acid sequence of any of SEQ ID NO: 1 through SEQ ID NO: 3. Alternatively, the binding region can have an amino acid sequence selected from the group consisting of: (1) the binding region of the amino acid sequence of any of SEQ ID NO: 1 through SEQ ID NO: 57, any of SEQ ID NO: 1 through SEQ ID NO: 10, or any of SEQ ID NO: 1 through SEQ ID NO: 3; and (2) a binding region differing from the amino acid sequence of any of SEQ ID NO: 1 through SEQ ID NO: 57, any of SEQ ID NO: 1 through SEQ ID NO: 10, or any of SEQ ID NO: 1 through SEQ ID NO: 3 by no more than two conservative amino acid substitutions, wherein the dissociation constant is no greater than 125% of that of the polypeptide before the substitutions are made, and wherein a conservative amino acid substitution is one of the following substitutions: Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. In still another alternative, the nucleotide binding region comprises a 7-amino acid zinc finger domain in which the seven amino acids of the domain are numbered from −1 to 6, and wherein the domain is selected from the group consisting of: (1) a zinc finger nucleotide binding domain specifically binding the nucleotide sequence 5′-(AGC)-3′, wherein the amino acid residue of the domain numbered −1 is selected from the group consisting of Q, N, S, G, H, and D; (2) a zinc finger nucleotide binding domain specifically binding the nucleotide sequence 5′-(AGC)-3′, wherein the amino acid residue of the domain numbered 3 is selected from the group consisting of W, T, and H; (3) a zinc finger nucleatide binding domain specifically binding the nucleotide sequence 5′-(AGC)-3′ wherein the amino acid residue of the domain numbered 4 is selected from the group consisting of L, V, I, and C; (4) a zinc finger nucleotide binding domain specifically binding the nucleotide sequence 5′-(AGC)-3′ wherein the amino acid residue of the domain numbered 6 is selected from the group consisting of A, R, N, D, Q, E, T, and V; and (5) a zinc finger nucleotide binding domain specifically binding the nucleotide sequence 5′-(AGC)-3′ wherein the amino acid residue of the domain numbered −1 is selected from the group consisting of D and E and wherein the residues of the domain numbering 4 through 6 are selected from the group consisting of LIN, LRE, and LTE.

In another aspect, the present invention provides a polypeptide composition that contains a plurality of and, preferably from about 2 to about 18 of zinc finger nucleotide binding domains as disclosed herein. The domains are typically operatively linked such as linked via a flexible peptide linker of from 5 to 15 amino acid residues. Operatively linked preferably occurs via a flexible peptide linker such as that shown in SEQ ID NO: 100 through SEQ ID NO: 107. Such a composition typically binds to a nucleotide sequence that contains a sequence of the formula 5′-(AGC)ₙ, 3′, where N is A, C, G or T and n is 2 to 12. Preferably, the polypeptide composition contains from about 2 to about 6 zinc finger nucleotide binding domains and binds to a nucleotide sequence that contains a sequence of the formula 5′-(AGC)ₙ-3′, where n is 2 to 6. Binding occurs with a K_{D} of from 1 μM to 10 μM. Preferably binding occurs with a K_{D} of from 10 μM to 1 μM, from 10 pM to 100 nM, from 100 pM to 10 nM and, more preferably with a K_{D} of from 1 nM to 10 nM. In preferred embodiments, both a polypeptide and a polypeptide composition of this invention are operatively linked to one or more transcription regulating factors such as a repressor of transcription or an activator of transcription.

In yet another aspect, the invention further provides an isolated heptapeptide having an α-helical structure and that binds preferentially to a target nucleotide of the formula AGC, The preferred heptapeptides are the same as those of the binding regions of the polypeptides described above.

Additionally, the invention further provides bispecific zinc fingers, the bispecific zinc fingers comprising two halves, each half comprising six zinc finger nucleotide binding domains, where at least one of the halves includes at least one domain binding a target nucleotide sequence of the form 5′-(AGC)-3′, such that the two halves of the bispecific zinc fingers can operate independently.

Additionally, the invention further provides a sequence-specific nuclease comprising the nuclease catalytic domain of Fokl, the sequence-specific nuclease cleaving at a site including therein at least one target nucleotide sequence of the form 5′-(AGC)-3′. The invention further provides methods for sequence-specific cleavage of nucleic acid sequences using such sequence-specific nucleases.

The present invention further provides polynucleotides that encode a polypeptide or a composition of this invention, expression vectors that contain such polynucleotides and host cells transformed with the polynucleotide or expression vector.

The present invention further provides a process of regulating expression of a nucleotide sequence that contains the target nucleotide sequence 5′-(AGC)-3′. The target nucleotide sequence can be located anywhere within a longer 5′-(NNN)-3′ sequence. The process includes the step of exposing the nucleotide sequence to an effective amount of a zinc finger nucleotide binding polypeptide or composition as set forth herein. In one embodiment, a process regulates expression of a nucleotide sequence that contains the sequence 5′-(AGC)ₙ-3′, where n is 2 to 12. The process includes the step of exposing the nucleotide sequence to an effective amount of a composition of this invention. The sequence 5′-(AGC)ₙ-3′ can be located in the transcribed region of the nucleotide sequence, in a promoter region of the nucleotide sequence, or within an expressed sequence tag. The composition is preferably operatively linked to one or more transcription regulating factors such as a repressor of transcription or an activator of transcription. In one embodiment, the nucleotide sequence is a gene such as a eukaryotic gene, a prokaryotic gene or a viral gene. The eukaryotic gene can be a mammalian gene such as a human gene, or, alternatively, a plant gene. The prokaryotic gene can be a bacterial gene.

In yet another embodiment, the invention provides a pharmaceutical composition comprising,

(1) a therapeutically effective amount of a polypeptide, polypeptide composition, or isolated heptapeptide according to the present invention as described above; and

(2) a pharmaceutically acceptable carrier.

In yet another embodiment, the invention provides a pharmaceutical composition comprising:

(1) a therapeutically effective amount of a nucleotide sequence that encodes a polypeptide, polypeptide composition, or isolated heptapeptide according to the present invention as described above; and

(2) a pharmaceutically acceptable carrier.

### BRIEF DESCRIPTION OF THE DRAWINGS

The following invention will become better understood with reference to the specification, appended claims, and accompanying drawings, where:

FIG. 1 is a model of the zinc finger-DNA complex of the murine transcription factor Zif268.

FIG. 2 shows, schematically, construction of the zinc finger phage display library. Solid arrows show interactions of the amino acid residues of the zinc finger helices with the nucleotides of their binding site as determined by x-ray crystallography of Zif268 and dotted lines show proposed interactions.

FIG. 3 is a diagram showing the structure and function of the linker region of the zinc finger protein Zif268.

FIG. 4 is a diagram showing a design concept for the construction of improved linkers (Example 3).

FIG. 5 is a series of graphs showing multitarget ELISA analysis of zinc finger domains produced by rational design and site-directed mutagenesis (ERS-H-LRE (SEQ ID NO: 2) and (DPG-H-LTE (SEQ ID NO: 3)).

### DETAILED DESCRIPTION OF THE INVENTION

Definitions

Unless defined otherwise, all technical and scientific terms used herein have the same meaning as is commonly understood by one of skill in the art to which this invention belongs.

As used herein, the term “nucleic acid,” “nucleic acid sequence,” “polynucleotide,” or similar terms, refers to a deoxyribonucleotide or ribonucleotide oligonucleotide or polynucleotide, including single- or double-stranded forms, and coding or non-coding (e.g., “antisense”) forms. The term encompasses nucleic acids containing known analogues of natural nucleotides. The term also encompasses nucleic acids including modified or substituted bases as long as the modified or substituted bases interfere neither with the Watson-Crick binding of complementary nucleotides or with the binding of the nucleotide sequence by proteins that bind specifically, such as zinc finger proteins. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992), Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methylphosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156).

As used herein, the term “transcription regulating domain or factor” refers to the portion of the fusion polypeptide provided herein that functions to regulate gene transcription. Exemplary and preferred transcription repressor domains are ERD, KRAB, SID, Deacetylase, and derivatives, multimers and combinations thereof such as KRAB-ERD, SID-ERD, (KRAB)₂, (KRAB)₃, KRAB-A, (KRAB-A)₂, (SID)₂, (KRAB-A)-SID and SID-(KRAB-A). As used herein, the term “nucleotide binding domain or region” refers to the portion of a polypeptide or composition provided herein that provides specific nucleic acid binding capability. The nucleotide binding region functions to target a subject polypeptide to specific genes. As used herein, the term “operatively linked” means that elements of a polypeptide, for example, are linked such that each performs or functions as intended. For example, a repressor is attached to the binding domain in such a manner that, when bound to a target nucleotide via that binding domain, the repressor acts to inhibit or prevent transcription. Linkage between and among elements may be direct or indirect, such as via a linker. The elements are not necessarily adjacent. Hence a repressor domain can be linked to a nucleotide binding domain using any linking procedure well known in the art. It may be necessary to include a linker moiety between the two domains. Such a linker moiety is typically a short sequence of amino acid residues that provides spacing between the domains. So long as the linker does not interfere with any of the functions of the binding or repressor domains, any sequence can be used.

As used herein, the term “modulating” envisions the inhibition or suppression of expression from a promoter containing a zinc finger-nucleotide binding motif when it is over-activated, or augmentation or enhancement of expression from such a promoter when it is underactivated.

As used herein, the amino acids, which occur in the various amino acid sequences appearing herein, are identified according to their well-known, three-letter or one-lefter abbreviations. The nucleotides, which occur in the various DNA fragments, are designated with the standard single-letter designations used routinely in the art.

In a peptide or protein, suitable conservative substitutions of amino acids are known to those of skill in this art and may be made generally without altering the biological activity of the resulting molecule. Those of skill in this art recognize that in general, single amino acid substitutions in non-essential regions of a polypeptide do not substantially alter biological activity (see, e.g. Watson et al. Molecular Biology of the Gene, 4th Edition, 1987, Benjamin/Cummings, p. 224). In particular, such a conservative variant has a modified amino acid sequence, such that the change(s) do not substantially alter the protein's (the conservative variant's) structure and/or activity, e.g., antibody activity, enzymatic activity, or receptor activity. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val, Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gln); (4) arginine (R or Arg), lysine (K or Lys); (5) isoleucine (I or Ile), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations” when the three-dimensional structure and the function of the protein to be delivered are conserved by such a variation.

As used herein, the term “expression vector” refers to a plasmid, virus, phagemid, or other vehicle known in the art that has been manipulated by insertion or incorporation of heterologous DNA, such as nucleic acid encoding the fusion proteins herein or expression cassettes provided herein. Such expression vectors typically contain a promoter sequence for efficient transcription of the inserted nucleic acid in a cell. The expression vector typically contains an origin of replication, a promoter, as well as specific genes that permit phenotypic selection of transformed cells.

As used herein, the term “host cells” refers to cells in which a vector can be propagated and its DNA expressed. The term also includes any progeny of the subject host cell. It is understood that all progeny may not be identical to the parental cell since there may be mutations that occur during replication. Such progeny are included when the term “host cell” is used. Methods of stable transfer where the foreign DNA is continuously maintained in the host are known in the art.

As used herein, genetic therapy involves the transfer of heterologous DNA to the certain cells, target cells, of a mammal, particularly a human, with a disorder or conditions for which such therapy is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product, or it may encode a product, such as a peptide or RNA that in some manner mediates, directly or indirectly, expression of a therapeutic product. Genetic therapy may also be used to deliver nucleic acid encoding a gene product that replaces a defective gene or supplements a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof. Genetic therapy may also involve delivery of an inhibitor or repressor or other modulator of gene expression.

As used herein, heterologous DNA is DNA that encodes RNA and proteins that are not normally produced in vivo by the cell in which it is expressed or that mediates or encodes mediators that alter expression of endogenous DNA by affecting transcription, translation, or other regulatable biochemical processes. Heterologous DNA may also be referred to as foreign DNA. Any DNA that one of skill in the art would recognize or consider as heterologous or foreign to the cell in which is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced.

Hence, herein heterologous DNA or foreign DNA, includes a DNA molecule not present in the exact orientation and position as the counterpart DNA molecule found in the genome. It may also refer to a DNA molecule from another organism or species (i.e., exogenous).

As used herein, a therapeutically effective product is a product that is encoded by heterologous nucleic acid, typically DNA, that, upon introduction of the nucleic acid into a host, a product is expressed that ameliorates or eliminates the symptoms, manifestations of an inherited or acquired disease or that cures the disease. Typically, DNA encoding a desired gene product is cloned into a plasmid vector and introduced by routine methods, such as calcium-phosphate mediated DNA uptake (see, (1981) Somat. Cell. Mol. Genet. 7:603-616) or microinjection, into producer cells, such as packaging cells. After amplification in producer cells, the vectors that contain the heterologous DNA are introduced into selected target cells.

As used herein, an expression or delivery vector refers to any plasmid or virus into which a foreign or heterologous DNA may be inserted for expression in a suitable host cell—i.e., the protein or polypeptide encoded by the DNA is synthesized in the host cell's system. Vectors capable of directing the expression of DNA segments (genes) encoding one or more proteins are referred to herein as “expression vectors”. Also included are vectors that allow cloning of cDNA (complementary DNA) from mRNAs produced using reverse transcriptase.

As used herein, a gene refers to a nucleic acid molecule whose nucleotide sequence encodes an RNA or polypeptide. A gene can be either RNA or DNA. Genes may include regions preceding and following the coding region (leader and trailer) as well as intervening sequences (introns) between individual coding segments (exons).

As used herein, the term “isolated” with reference to a nucleic acid molecule or polypeptide or other biomolecule means that the nucleic acid or polypeptide has been separated from the genetic environment from which the polypeptide or nucleic acid were obtained. It may also mean that the biomolecule has been altered from the natural state. For example, a polynucleotide or a polypeptide naturally present in a living animal is not “isolated,” but the same polynucleotide or polypeptide separated from the coexisting materials of its natural state is “isolated,” as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compound can be substantially purified by the one-step method described in Smith et al. (1988) Gene 67:3140. The terms isolated and purified are sometimes used interchangeably.

Thus, by “isolated” is meant that the nucleic acid is free of the coding sequences of those genes that, in a naturally-occurring genome immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

“Isolated” or “purified” as those terms are used to refer to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. Particularly for proteins, the procedures may include for example, but are not limited to, ammonium sulfate fractionation, gel filtration, ion exchange change chromatography, affinity chromatography, density gradient centrifugation, electrofocusing, chromatofocusing, and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, “modulate” refers to the suppression, enhancement or induction of a function. For example, zinc finger-nucleic acid binding domains and variants thereof may modulate a promoter sequence by binding to a motif within the promoter, thereby enhancing or suppressing transcription of a gene operatively linked to the promoter cellular nucleotide sequence. Alternatively, modulation may include inhibition of transcription of a gene where the zinc finger-nucleotide binding polypeptide variant binds to the structural gene and blocks DNA dependent RNA polymerase from reading through the gene, thus inhibiting transcription of the gene. The structural gene may be a normal cellular gene or an oncogene, for example. Alternatively, modulation may include inhibition of translation of a transcript.

As used herein, the term “inhibit” refers to the suppression of the level of activation of transcription of a structural gene operably linked to a promoter. For example, for the methods herein the gene includes a zinc finger-nucleotide binding motif.

As used herein, the term “transcriptional regulatory region” refers to a region that drives gene expression in the target cell. Transcriptional regulatory regions suitable for use herein include but are not limited to the human cytomegalovirus (CMV) immediate-early enhancer/promoter, the SV40 early enhancer/promoter, the JC polyoma virus promoter, the albumin promoter, PGK and the a-actin promoter coupled to the CMV enhancer. Other transcriptional regulatory regions are also known in the art.

As used herein, a promoter region of a gene includes the regulatory element or elements that typically lie 5′ to a structural gene; multiple regulatory elements can be present, separated by intervening nucleotide sequences. If a gene is to be activated, proteins known as transcription factors attach to the promoter region of the gene. This assembly resembles an “on switch” by enabling an enzyme to transcribe a second genetic segment from DNA into RNA. In most cases the resulting RNA molecule serves as a template for synthesis of a specific protein; sometimes RNA itself is the final product. The promoter region may be a normal cellular promoter or, for example, an onco-promoter. An onco-promoter is generally a virus-derived promoter. Viral promoters to which zinc finger binding polypeptides may be targeted include, but are not limited to, retroviral long terminal repeats (LTRs), and Lentivirus promoters, such as promoters from human T-cell lymphotrophic virus (HTLV) 1 and 2 and human immunodeficiency virus (HIV) 1 or 2.

As used herein, the term “effective amount” includes that amount that results in the deactivation of a previously activated promoter or that amount that results in the inactivation of a promoter containing a zinc finger-nucleotide binding motif, or that amount that blocks transcription of a structural gene or translation of RNA. The amount of zinc finger derived-nucleotide binding polypeptide required is that amount necessary to either displace a native zinc finger-nucleotide binding protein in an existing protein/promoter complex, or that amount necessary to compete with the native zinc finger-nucleotide binding protein to form a complex with the promoter itself. Similarly, the amount required to block a structural gene or RNA is that amount which binds to and blocks RNA polymerase from reading through on the gene or that amount which inhibits translation, respectively. Preferably, the method is performed intracellularly. By functionally inactivating a promoter or structural gene, transcription or translation is suppressed. Delivery of an effective amount of the inhibitory protein for binding to or “contacting” the cellular nucleotide sequence containing the zinc finger-nucleotide binding protein motif, can be accomplished by one of the mechanisms described herein, such as by retroviral vectors or liposomes, or other methods well known in the art.

As used herein, the term “truncated” refers to a zinc finger-nucleotide binding polypeptide derivative that contains less than the full number of zinc fingers found in the native zinc finger binding protein or that has been deleted of non-desired sequences. For example, truncation of the zinc finger-nucleotide binding protein TFIIIA, which naturally contains nine zinc fingers, might result in a polypeptide with only zinc fingers one through three. The term “expansion” refers to a zinc finger polypeptide to which additional zinc finger modules have been added. For example, TFIIIA can be expanded to 12 fingers by adding 3 zinc finger domains. In addition, a truncated zinc finger-nucleotide binding polypeptide may include zinc finger modules from more than one wild type polypeptide, thus resulting in a “hybrid” zinc finger-nucleotide binding polypeptide.

As used herein, the term “mutagenized” refers to a zinc finger derived-nucleotide binding polypeptide that has been obtained by performing any of the known methods for accomplishing random or site-directed mutagenesis of the DNA encoding the protein. For instance, in TFIIIA, mutagenesis can be performed to replace nonconserved residues in one or more of the repeats of the consensus sequence. Truncated or expanded zinc finger-nucleotide binding proteins can also be mutagenized.

As used herein, a polypeptide “variant” or “derivative” refers to a polypeptide that is a mutagenized form of a polypeptide or one produced through recombination but that still retains a desired activity, such as the ability to bind to a ligand or a nucleic acid molecule or to modulate transcription.

As used herein, a zinc finger-nucleotide binding polypeptide “variant” or “derivative” refers to a polypeptide that is a mutagenized form of a zinc finger protein or one produced through recombination. A variant may be a hybrid that contains zinc finger domain(s) from one protein linked to zinc finger domain(s) of a second protein, for example. The domains may be wild type or mutagenized. A “variant” or “derivative” can include a truncated form of a wild type zinc finger protein, which contains fewer than the original number of fingers in the wild type protein. Examples of zinc finger-nucleotide binding polypeptides from which a derivative or variant may be produced include TFIIIA and zif268. Similar terms are used to refer to “variant” or “derivative” nuclear hormone receptors and “variant” or “derivative” transcription effector domains.

As used herein a “zinc finger-nucleotide binding target or motif” refers to any two or three-dimensional feature of a nucleotide segment to which a zinc finger-nucleotide binding derivative polypeptide binds with specificity. Included within this definition are nucleotide sequences, generally of five nucleotides or less, as well as the three dimensional aspects of the DNA double helix, such as, but are not limited to, the major and minor grooves and the face of the helix. The motif is typically any sequence of suitable length to which the zinc finger polypeptide can bind. For example, a three finger polypeptide binds to a motif typically having about 9 to about 14 base pairs. Preferably, the recognition sequence is at least about 16 base pairs to ensure specificity within the genome. Therefore, zinc finger-nucleotide binding polypeptides of any specificity are provided. The zinc finger binding motif can be any sequence designed empirically or to which the zinc finger protein binds. The motif may be found in any DNA or RNA sequence, including regulatory sequences, exons, introns, or any non-coding sequence.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like which would be to a degree that would prohibit administration of the composition.

As used herein, the term “vector” refers to a nucleic acid molecule capable of transporting between different genetic environments another nucleic acid to which it has been operatively linked. Preferred vectors are those capable of autonomous replication and expression of structural gene products present in the DNA segments to which they are operatively linked. Vectors, therefore, preferably contain the replicons and selectable markers described earlier. Vectors include, but are not necessarily limited to, expression vectors.

As used herein with regard to nucleic acid molecules, including DNA fragments, the phrase “operatively linked” means the sequences or segments have been covalently joined, preferably by conventional phosphodiester bonds, into one strand of DNA, whether in single or double-stranded form such that operatively linked portions function as intended. The choice of vector to which transcription unit or a cassette provided herein is operatively linked depends directly, as is well known in the art, on the functional properties desired, e.g., vector replication and protein expression, and the host cell to be transformed, these being limitations inherent in the art of constructing recombinant DNA molecules.

As used herein, administration of a therapeutic composition can be effected by any means, and includes, but is not limited to, oral, subcutaneous, intravenous, intramuscular, intrasternal, infusion techniques, intraperitoneal administration and parenteral administration.

I. The Invention

The present invention provides zinc finger-nucleotide binding polypeptides, compositions containing one or more such polypeptides, polynucleotides that encode such polypeptides and compositions, expression vectors containing such polynucleotides, cells transformed with such polynucleotides or expression vectors and the use of the polypeptides, compositions, polynucleotides and expression vectors for modulating nucleotide structure and/or function.

II. Polypeptides

The present invention provides an isolated and purified zinc finger nucleotide binding polypeptide. The polypeptide contains a nucleotide binding region of from 5 to 10 amino acid residues and, preferably about 7 amino acid residues. Typically, the nucleotide binding region is a sequence of seven amino acids, referred to herein as a “domain,” that is predominantly α-helical in its conformation. The structure of this domain is described below in further detail. However, the nucleotide binding region can be flanked by up to five amino acids on each side and the term “domain,” as used herein, includes these additional amino acids. The nucleotide binding region binds preferentially to a target nucleotide of the formula AGC.

A polypeptide of this invention is a non-naturally occurring variant. As used herein, the term non-naturally occurring means, for example, one or more of the following: (a) a polypeptide comprised of a non-naturally occurring amino acid sequence; (b) a polypeptide having a non-naturally occurring secondary structure not associated with the polypeptide as it occurs in nature; (c) a polypeptide which includes one or more amino acids not normally associated with the species of organism in which that polypeptide occurs in nature; (d) a polypeptide which includes a stereoisomer of one or more of the amino acids comprising the polypeptide, which stereoisomer is not associated with the polypeptide as it occurs in nature; (e) a polypeptide which includes one or more chemical moieties other than one of the natural amino acids; or (f) an isolated portion of a naturally occurring amino acid sequence (erg., a truncated sequence). A polypeptide of this invention exists in an isolated form and purified to be substantially free of contaminating substances. The polypeptide can be isolated and purified from natural sources; alternatively, the polypeptide can be made de novo using techniques well known in the art such as genetic engineering or solid-phase peptide synthesis. A zinc finger-nucleotide binding polypeptide refers to a polypeptide that is, preferably, a mutagenized form of a zinc finger protein or one produced through recombination. A polypeptide may be a hybrid which contains zinc finger domain(s) from one protein linked to zinc finger domain(s) of a second protein, for example. The domains may be wild type or mutagenized. A polypeptide can include a truncated form of a wild type zinc finger protein. Examples of zinc finger proteins from which a polypeptide can be produced include SP1C, TFIIIA and Zif268, as well as C7 (a derivative of Zif268) and other zinc finger proteins known in the art. These zinc finger proteins from which other zinc finger proteins are derived are referred to herein as “backbones.”

A zinc finger-nucleotide binding polypeptide of this invention comprises a unique heptamer (contiguous sequence of 7 amino acid residues) within the α-helical domain of the polypeptide, which heptameric sequence determines binding specificity to a target nucleotide. That heptameric sequence can be located anywhere within the α-helical domain but it is preferred that the heptamer extend from position −1 to position 6 as the residues are conventionally numbered in the art. A polypeptide of this invention can include any β-sheet and framework sequences known in the art to function as part of a zinc finger protein. A large number of zinc finger-nucleotide binding polypeptides were made and tested for binding specificity against target nucleotides containing an AGC triplet.

The zinc finger-nucleotide binding polypeptide derivative can be derived or produced from a wild type zinc finger protein by truncation or expansion, or as a variant of the wild type-derived polypeptide by a process of site directed mutagenesis, or by a combination of the procedures. In addition, a truncated zinc finger-nucleotide binding polypeptide may include zinc finger modules from more than one wild type polypeptide, thus resulting in a “hybrid”, zinc finger-nucleotide binding polypeptide.

The term “mutagenized” refers to a zinc finger derived-nucleotide binding polypeptide that has been obtained by performing any of the known methods for accomplishing random or site-directed mutagenesis of the DNA encoding the protein. For instance, in TFIIIA, mutagenesis can be performed to replace nonconserved residues in one or more of the repeats of the consensus sequence. Truncated zinc finger-nucleotide binding proteins can also be mutagenized. Examples of known zinc finger-nucleotide binding polypeptides that can be truncated, expanded and/or mutagenized according to the present invention in order to inhibit the function of a nucleotide sequence containing a zinc finger-nucleotide binding motif includes TFIIIA and zif268. Those of skill in the art know other zinc finger-nucleotide binding proteins.

Typically, the binding region has seven amino acid residues and has α-helical structure.

In addition, the polypeptides of the present invention can be incorporated within longer polypeptides. Some examples of this are described below, when the polypeptides are used to create artificial transcription factors. In general, though the polypeptides can be incorporated into longer fusion proteins and retain their specific DNA binding activity. These fusion proteins can include various additional domains as are known in the art, such as purification tags, enzyme domains, or other domains, without significantly altering the specific DNA-binding activity of the zinc finger polypeptides. In one example, the polypeptides can be incorporated into two halves of a split enzyme like a β-lactamase to allow the sequences to be sensed in cells or in vivo. Binding of two halves of such a split enzyme then allows for assembly of the split enzyme (J. M. Spotts et al. “Time-Lapse Imaging of a Dynamic Phosphorylation Protein-Protein Interaction in Mammalian Cells,” Proc. Natl. Acad. Sci. USA 99: 15142-15147 (2002)). In another example, multiple zinc finger domains according to the present invention can be tandemly linked to form polypeptides that have specific binding affinity for longer DNA sequences. This is described further below.

A polypeptide of this invention can be made using a variety of standard techniques well known in the art. As disclosed in detail hereinafter in the Examples, phage display libraries of zinc finger proteins were created and selected under conditions that favored enrichment of sequence specific proteins. Zinc finger domains recognizing a number of sequences required refinement by site-directed mutagenesis that was guided by both phage selection data and structural information.

Previously we reported the characterization of 16 zinc finger domains specifically recognizing each of the 5′-(GNN)-3′ type of DNA sequences, that were isolated by phage display selections based on C7, a variant of the mouse transcription factor Zif268 and refined by site-directed mutagenesis [Segal et al., (1999) Proc Natl Acad Sci USA 96(6), 2758-2763; Dreier et al., (2000) J. Mol. Biol. 303, 489-502; and U.S. Pat. No. 6,140,081, the disclosure of which is incorporated herein by reference]. In general, the specific DNA recognition of zinc finger domains of the Cys₂-His₂ type is mediated by the amino acid residues −1, 3, and 6 of each α-helix, although not in every case are all three residues contacting a DNA base. One dominant cross-subsite interaction has been observed from position 2 of the recognition helix. Asp² has been shown to stabilize the binding of zinc finger domains by directly contacting the complementary adenine or cytosine of the 5′ thymine or guanine, respectively, of the following 3 bp subsite. These non-modular interactions have been described as target site overlap. In addition, other interactions of amino acids with nucleotides outside the 3 bp subsites creating extended binding sites have been reported [Pavletich et al., (1991 ) Science 252(5007), 809-817; Elrod-Erickson et al., (1996) Structure 4(10), 1171-1180; Isalan et al., (1997) Proc Natl Acad Sci USA 94(11), 5617-5621].

Some of the generalizations of sequences of zinc finger domains binding particular DNA triplets obtained from results on a large number of zinc finger domains are shown in Table 1, below. In general, the −1-amino acid of a zinc finger domain is primarily responsible for the specification of the 3′-nucleotide of a triplet site, the 3-amino acid of a zinc finger domain is primarily responsible for the specification of the middle nucleotide of a triplet site, and the 6-amino acid of a zinc finger domain is primarily responsible for the specification of the 5′-nucleotide of a triplet site. These generalizations are used below to construct additional zinc fingers based on the zinc fingers that are described in Example 1.

TABLE 1
Protein/DNA-Interactions of Zinc finger domains (D.
J. Segal, B. Dreier, R. R. Beerli, C. F. Barbas III,
Proc. Natl. Acad. Sci. USA 1999, 96, 2758-2763.)

| Position within | | | |
|---|---|---|---|
| the triplet | | | |
| Nucleotide | 5′ | Middle | 3′ |
| Adenine | nd | Asn | Gln |
| Cytosine | nd | Thr, Asp, Glu | Asp, Glu |
| Guanine | Arg | His, Lys | Arg |
| Thymine | nd | Ser, Ala | Thr, Ser |

Selection of the previously reported phage display library for zinc finger domains binding to 5′ nucleotides other than guanine or thymine met with no success, due to the cross-subsite interaction from aspartate in position 2 of the finger-3 recognition helix RSD-E-LKR (SEQ ID NO: 58). To extend the availability of zinc finger domains for the construction of artificial transcription factors, domains specifically recognizing the 5′-(ANN)-3′ type of DNA sequences were selected (U.S. patent application Ser. No. 09/791,106, filed Feb. 21, 2001, the disclosure of which is incorporated herein by reference). Other groups have described a sequential selection method which led to the characterization of domains recognizing four 5′-(ANN)-3′ subsites, 5′-(AAA)-3′, 5′-(AAG)-3′, 5′-(ACA)-3′, and 5′-(ATA)-3′ [Greisman et al., (1997) Science 275(5300), 657-661; Wolfe et al., (1999) J Mol Biol 285(5), 1917-1934]. The present disclosure uses an approach to select zinc finger domains recognizing AGC sites by eliminating the target site overlap.

Based on the 3-finger protein C7.GAT, a library was previously constructed in the phage display vector pComb3H [Barbas et al., (1991) Proc. Natl. Acad. Sci. USA 88, 7978-7982; Rader et al., (1997) Curr. Opin. Biotechnol. 8(4), 503-508]. Randomization involved positions −1, 1, 2, 3, 5, and 6 of the α-helix of finger 2 using a VNS codon doping strategy (V=adenine, cytosine or guanine, N=adenine, cytosine, guanine or thymine, S=cytosine or guanine). This allowed 24 possibilities for each randomized amino acid position, whereas the aromatic amino acids Trp, Phe, and Tyr, as well as stop codons, were excluded in this strategy. Because Leu is predominately found in position 4 of the recognition helices of zinc finger domains of the type Cys₂-His₂ this position was not randomized. After transformation of the library into ER2537 cells (New England Biolabs) the library contained 1.5×10⁹ members. This exceeded the necessary library size by 60-fold and was sufficient to contain all amino acid combinations.

Previously, with respect to zinc finger domains binding sequences of the form 5′-(CNN)-3′, six rounds of selection of zinc finger-displaying phage were performed binding to each of the sixteen 5′-GAT-CNN-GCG-3′ (SEQ ID NO: 109) biotinylated hairpin target oligonucleotides, respectively, in the presence of non-biotinylated competitor DNA. Stringency of the selection was increased in each round by decreasing the amount of biotinylated target oligonucleotide and increasing amounts of the competitor oligonucleotide mixtures. In the sixth round the target concentration was usually 18 nM, 5′-(ANN)-3′, 5′-(GNN)-3′, and 5′-(TNN)-3′ competitor mixtures were in 5-fold excess for each oligonuclebtide pool, respectively, and the specific 5′-(CNN)-3′ mixture (excluding the target sequence) in 10-fold excess. Phage binding to the biotinylated target oligonucleotide was recovered by capture to streptavidin-coated magnetic beads. Clones were usually analyzed after the sixth round of selection. A similar selection process can be used for the selection of zinc finger domains binding specifically to sequences of the form 5′-(AGC)-3′. This process is described below in Example 1.

The amino acid sequences of selected finger-2 helices were determined and generally showed good conservation in positions −1 and 3, consistent with previously observed amino acid residues in these positions [Segal et al., (1999) Proc Natl Acad Sci USA 96(6), 2758-2763]. Position −1 was Gln when the 3′ nucleotide was adenine, with the exception of domains binding 5′-ACA-3′ (SPA-D-LTN) (SEQ ID NO: 59) where a Ser was strongly selected. Triplets containing a 3′ cytosine selected Asp⁻¹ (exceptions were domains binding 5′-AGC-3′ and 5′-ATC-3′), a 3′ guanine Arg⁻¹, and a 5′ thymine Thr⁻¹ and His⁻¹. The recognition of a 3′ thymine by His⁻¹ has also been observed in finger 1 of TKK binding to 5′-GAT-3′ (HIS-N-FCR) (SEQ ID NO: 60); [Fairall et al., (1993) Nature (London) 366(6454), 483-7]). For the recognition of a middle adenine, Asp and Thr were selected in position 3 of the recognition helix. For binding to a middle cytosine, an Asp³ or Thr³ was selected, for a middle guanine, His³ (an exception was recognition of 5′-AGT-3′, which may have a different binding mechanism due to the unusual amino acid residue His⁻¹) and for a middle thymine, Ser³ and Ala³. Note also that the domains binding to 5′-ANG-3′ subsites contain Asp² which likely stabilizes the interaction of the 3-finger protein by contacting the complementary cytosine of the 5′ guanine in the finger-1 subsite. Even though there was a predominant selection of Arg and Thr in position 5 of the recognition helices, positions 1, 2 and 5 were variable.

The most interesting observation was the selection of amino acid residues in position 6 of the α-helices that determines binding to the 5′ nucleotide of a 3 bp subsite. In contrast to the recognition of a 5′ guanine, where the direct base contact is achieved by Arg or Lys in position 6 of the helix, no direct interaction has been observed in protein/DNA complexes for any other nucleotide in the 5′ position [Elrod-Erickson et al., (1996) Structure 4(10), 1171-1180; Pavletich et al., (1993) Science (Washington, D.C., 1883-) 261(5129), 1701-7; Kim et al., (1996) Nat Struct Biol 3(11), 940-945; Fairall et al., (1993) Nature (London) 366(6454), 483-7; Houbaviy et al., (1996) Proc Natl Acad Sci USA 93(24), 13577-82; Wutike et al., (1997) J Mol Biol 273(1), 183-206; Nolte et al., (1998) Proc Natl Acad Sci USA 95(6), 2938-2943]. Selection of domains against finger-2 subsites of the type 5′-GNN-3′ had previously generated domains containing only Arg⁶ which directly contacts the 5′ guanine [Segal et al., (1999) Proc Natl Acad Sci USA 96(6), 2758-2763]. However, unlike the results for 5′-GNN-3′ zinc finger domains, selections of the phage display library against finger-2 subsites of the type 5′-ANN-3′ identified domains containing various amino acid residues: Ala⁶, Arg⁶, Asn⁶, Asp⁶, Gln⁶, Glu⁶, Thr⁶ or Val6. In addition, one domain recognizing 5′-TAG-3′ was selected from this library with the amino acid sequence RED-N-LHT (SEQ ID NO: 61). Thr⁶ is also present in finger 2 of Zif268 (RSD-H-LTT) (SEQ ID NO: 62) binding 5′-TGG-3′ for which no direct contact was observed in the Zif268/DNA complex.

Finger-2 variants of C7.GAT were subcloned into bacterial expression vector as fusion with maltose-binding protein (MBP) and proteins were expressed by induction with 1 mM IPTG (proteins (p) are given the name of the finger-2 subsite against which they were selected). Proteins were tested by enzyme-linked immunosorbent assay (ELISA) against each of the 16 finger-2 subsites of the type 5′-GAT ANN GCG-3′ (SEQ ID NO: 110) to investigate their DNA-binding specificity. In addition, the 5′-nucleotide recognition was analyzed by exposing zinc finger proteins to the specific target oligonucleotide and three subsites which differed only in the 5′-nucleotide of the middle triplet. For example, pAAA was tested on 5′-AAA-3′, 5′-CAA-3′, 5′-GAA-3′, and 5′-TAA-3′ subsites. Many of the tested 3-finger proteins showed exquisite DNA-binding specificity for the finger-2 subsite against which they were selected. The exceptions were pAGC and pATC whose DNA binding was too weak to be detected by ELISA. The most promising helix for pAGC (DAS-H-LHT) (SEQ ID NO: 63) obtained at this stage without further mutagenesis, which contained the expected amino acid Asp⁻¹ and His³ specifying a 3′ cytosine and middle guanine, but also a Thr⁶ not selected in any other case for a 5′ adenine, was analyzed without detectable DNA binding.

To analyze a larger set, the pool of coding sequences for pAGC was subcloned into the plasmid pMal after the sixth round of selection. Rational design was applied to find domains binding to 5′-AGC-3′ or 5′-ATC-3′, since no proteins binding these finger-2 subsites were generated by phage display. Finger-2 mutants were constructed based on the recognition helices which were previously demonstrated to bind specifically to 5′-GGC-3′ (ERS-K-LAR (SEQ ID NO: 64), DPG-H-LVR (SEQ ID NO: 65)) and 5′-GTC-3′ (DPG-A-LVR) (SEQ ID NO: 66) [Segal et al., (1999) Proc Natl Acad Sci USA 96(6), 2758-2763]. For pAGC two proteins were constructed (ERS-K-LRA (SEQ ID NO: 67), DPG-H-LRV (SEQ ID NO: 68)) by simply exchanging position 5 and 6 to a 5′ adenine recognition motif RA or RV. However, DNA binding of these proteins was below detection level. As detailed below, additional zinc finger domains capable of binding 5′-AGC-3′ have now been isolated and are described further. In the case of pATC two finger-2 mutants containing a RV motif were constructed (DPG-A-LRV (SEQ ID NO: 69), DPG-S-LRV (SEQ ID NO: 70)). Both proteins bound DNA with extremely low affinity regardless if position 3 was Ala or Ser.

Analysis of the 3-finger proteins on the sixteen finger-2 subsites by ELISA revealed that some finger-2 domains bound best to a target they were not selected against. First, the predominantly selected helix for 5′-AGA-3′ was RSD-H-LTN (SEQ ID NO: 71)), which in fact bound 5′-AGG-3′. This can be explained by the Arg in position −1. In addition, this protein showed a better discrimination of a 5′ adenine compared to the predominantly selected helix pAGG (RSD-H-LAE (SEQ ID NO: 72)). Second, a helix binding specifically to 5′-AAG-3′ (RSD-N-LKN (SEQ ID NO. 73)) was actually selected against 5′-AAC-3′, and bound more specifically to the finger-2 subsite 5′-AAG-3′ than PAAG (RSD-T-LSN (SEQ ID NO: 74)), which had been selected in the 5′-AAG-3′ set. In addition, proteins directed to target sites of the type 5′-ANG-3′ showed cross reactivity with all four target sites of the type 5′-ANG-3′, except for pAGG. The recognition of a middle purine seems more restrictive than of a middle pyrimidine, because also pAAG (RSD-N-LKN (SEQ ID NO: 73)) had only moderate cross-reactivity.

In comparison, the proteins pACG (RTD-T-LRD (SEQ ID NO: 75) and pATG (RRD-A-LNV (SEQ ID NO: 76);) show cross-reactivity with all 5′-ANG-3′ subsites. The recognition of a middle pyrimidine has been reported to be difficult in previous studies for domains binding to 5′-GNG-3′ DNA sequences [Segal et al., (1999) Proc Natl Acad Sci USA 96(6), 2758-2763; Dreier et al., (2000) J. Mol. Biol. 303, 489-502]. To improve the recognition of the middle nucleotide, finger-2 mutants containing different amino acid residues in position 3 were generated by site-directed mutagenesis. Binding of pAAG (RSD-T-LSN (SEQ ID NO: 74)) was more specific for a middle adenine after a Thr³ to Asn³ mutation. The binding to 5′-ATG-3′ (SRD-A-LNV (SEQ ID NO: 77)) was improved by a single amino acid exchange Ala³ to Gln³, while a Thr³ to Asp³ or Gln³ mutation for pACG (RSD-T-LRD (SEQ ID NO: 78)) abolished DNA binding. In addition, the recognition helix pAGT (HRT-T-LLN (SEQ ID NO: 79)) showed cross-reactivity for the middle nucleotide which was reduced by a Leu⁵ to Thr⁵ substitution. Surprisingly, improved discrimination for the middle nucleotide was often associated with some loss of specificity for the recognition of the 5′ adenine.

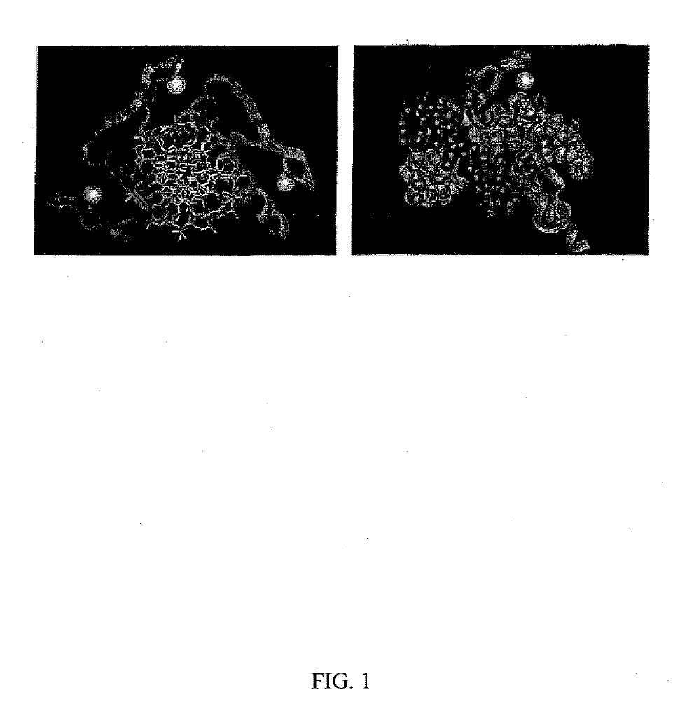

Selection of zinc finger domains binding to subsites containing a 5′ adenine or cytosine from the previously described finger-2 library based on the 3-finger protein C7 [Segal et al., (1999) Proc Natl Acad Sci USA 96(6), 2758-2763] was not suitable for the selection of zinc-finger domains due to the limitation of aspartate in position 2 of finger 3 which makes a cross-subsite contact to the nucleotide complementary of the 5′ position of the finger-2 subsite (FIG. 1a, upper panel). We eliminated this contact by exchanging finger 3 with a domain lacking Asp² (FIG. 1b). Finger 2 of C7.GAT was randomized and a phage display library constructed. In most cases, novel 3-finger proteins were selected binding to finger-2 subsites of the type 5′-ANN-3′. For the subsites 5′-AGC-3′ and 5′-ATC-3′ no tight binders were identified. This was not expected, because the domains binding to the subsite 5′-GGC-3′ and 5′-GTC-3′ previously selected from the C7-based phage display library showed excellent DNA-binding specificity and affinity of 40 nM to their target site [Segal et al., (1999) Proc Natl Acad Sci USA 96(6), 2758-2763]. One simple explanation would be the limiting randomization strategy by the usage of VNS codons which do not include the aromatic amino acid residues. These were not included in the library, because for the domains binding to 5′-GNN-3′ subsites no aromatic amino acid residues were selected, even though they were included in the randomization strategy [Segal et al., (1999) Proc Natl Acad Sci USA 96(6), 2758-2763]. However, there have been zinc finger domains reported containing aromatic residues, like finger 2 of CFII2 (VKD-Y-LTK (SEQ ID NO: 80); [Gogos et al., (1996) PNAS 93, 2159-2164]), finger 1 of TFIIIA (KNW-K-LQA (SEQ ID NO: 81; [Wuttke et al., (1997) J Mol Biol 273(1), 183-206]), finger 1 of TTK (HIS-N-FCR (SEQ ID NO: 82); [Fairall et al., (1993) Nature (London) 366(6454), 483-7]) and finger 2 of GLI (AQY-M-LVV (SEQ ID NO, 83); [Pavletich et al., (1993) Science (Washington, D. C., 1883-) 261(5129), 1701-7]). Aromatic amino acid residues might be important for the recognition of the subsites 5′-AGC-3′ and 5′-ATC-3′.

In recent years it has become clear that the recognition helix of Cys₂-His₂ zinc finger domains can adopt different orientations relative to the DNA in order to achieve optimal binding [Pabo et al., (2000) J. Mol. Biol. 301, 597-624]. However, the orientation of the helix in this region may be partially restricted by the frequently observed interaction involving the zinc ion, His⁷, and the phosphate backbone, Furthermore, comparison of binding properties of interactions in protein/DNA complexes have led to the conclusion that the Ca atom of position 6 is usually 8.8±0.8 Å apart from the nearest heavy atom of the 5′ nucleotide in the DNA subsite, which favors only the recognition of a 5′ guanine by Arg⁶ or Lys⁶ [Pabo et al., (2000) J. Mol. Biol. 301, 597-624]. To date, no interaction of any other position 6 residue with a base other than guanine has been observed in protein/DNA complexes. For example, finger 4 of YY1 (QST-N-LKS) (SEQ ID NO: 84) recognizes 5′-CAA-3′ but there was no contact observed between Ser⁶ and the 5′ cytosine [Houbaviy et al., (1996) Proc Natl Acad Sci USA 93(24), 13577-82]. Further, in the case of Thr⁶ in finger 3 of YY1 (LDF-N-LRT) (SEQ ID NO: 85), recognizing 5′-ATT-3′, and in finger 2 of Zif268 (RSD-H-LTT) (SEQ ID NO: 86), specifying 5′-T/GGG-3′, no contact with the 5′ nucleotide was observed [Houbaviy et al., (1996) Proc Natl Acad Sci USA 93(24), 13577-82; Elrod-Erickson et al., (1996) Structure 4(10), 1171-1180]. Finally, Ala⁶ of finger 2 of Tramtrack (RKD-N-MTA) (SEQ ID NO: 87) binding to the subsite 5′-AAG-3′ does not contact the 5′ adenine [Fairall et al., (1993) Nature (London) 366(6454), 483-7].

Amino acid residues Ala⁶, Val⁶, Asn⁶ and even Arg⁶, which in a different context was demonstrated to bind a 5′ guanine efficiently [Segal et al., (1999) Proc Natl Acad Sci USA 96(6), 2758-2763], were predominantly selected from the C7.GAT library for DNA subsites of the type 5′-ANN-3′. In addition, position 6 was selected as Thr, Glu and Asp depending on the finger-2 target site. This is consistent with early studies from other groups where positions of adjacent fingers were randomized [Jamieson et al., (1996) Proc Natl Acad Sci USA 93, 12834-12839; Isalan et al., (1998) Biochemistry 37(35), 12026-12033]. Screening of phage display libraries had resulted in selection of amino acid residues Tyr, Val, Thr, Asn, Lys, Glu and Leu, as well as Gly, Ser and Arg, but not Ala, for the recognition of a 5′ adenine. In addition, using a sequential phage display selection strategy several domains binding to 5′-ANN-3′ subsites were identified and specificity evaluated by target site selections. Arg, Ala and Thr in position 6 of the helix were demonstrated to recognize predominantly a 5′ adenine [Wolfe et al., (1999) Annu. Rev. Biophys. Biomol. Struct. 3, 183-212].

In addition, Thr⁶ specifies a 5′ adenine as shown by target site selection for finger 5 of Gfi-1 (QSS-N-LIT) (SEQ ID NO: 88) binding to the subside 5′-AAA-3′ [Zweidler-McKay et al., (1996) Mol. Cell. Biol. 16(8), 4024-4034]. These examples, including the present results, indicate that there is likely a relation between amino acid residue in position 6 and the 5′ adenine, because they are frequently selected. This is at odds with data from crystallographic studies, that never showed interaction of position 6 of the α-helix with a 5′ nucleotide except guanine. One simple explanation might be that short amino acid residues, like Ala, Val, Thr, or Asn are not a steric hindrance in the binding mode of domains recognizing 5′-ANN-3′ subsites. This is supported by results gathered by site-directed mutagenesis in position 6 for a helix (QRS-A-LTV) (SEQ ID NO: 89) binding to a 5′-G/ATA-3′ subsite [Gogos et al., (1996) PNAS 93, 2159-2164]. Replacement of Val⁶ with Ala⁶, which were also found for domains described here, or Lys⁶, had no effect on the binding specificity or affinity.

Computer modeling was used to investigate possible interactions of the frequently selected Ala⁶, Asn⁶ and Arg⁶ with a 5′ adenine. Analysis of the interaction from Ala⁶ in the helix binding to 5′-AAA-3′ (QRA-N-LRA) (SEQ ID NO: 90) with a 5′ adenine was based on the coordinates of the protein/DNA complex of finger 1 (QSG-S-LTR) (SEQ ID NO: 91) from a Zif268 variant. If Gln⁻¹ and Asn³ of QRA-N-LRA (SEQ ID NO: 90) hydrogen bond with their respective adenine bases in the canonical way, these interactions should fix a distance of about 8 Å between the methyl group of Ala⁶ and the 5′ adenine and more than 11 Å between the methyl groups of Ala⁶ and the thymine base-paired to the adenine, suggesting also that no direct contact can be proposed for Val⁶ and Thr⁶.

Interestingly, the expected lack of 5′ specificity by short amino acids in position 6 of the α-helix is only partially supported by the binding data. Helices such as RRD-A-LNV (SEQ ID NO: 76) and the finger-2 helix RSD-H-LTT (SEQ ID NO: 62) of C7.GAT did indeed show essentially no 5′ specificity. However, helix DSG-N-LRV (SEQ ID NO: 92) displayed excellent specificity for a 5′ adenine, while TSH-G-LTT (SEQ ID NO: 93) was specific for 5′ adenine or guanine. Other helices with short position-6 residues displayed varying degrees of 5′ specificity, with the only obvious consistency being that 5′ thymine was usually excluded. Since it is unlikely that the position-6 residue can make a direct contribution to specificity, the observed binding patterns must derive from another source. Possibilities include local sequence-specific DNA structure and overlapping interactions from neighboring domains. The latter possibility is disfavored, however, because the residue in position 2 of finger 3 (which is frequently observed to contact the neighboring site) is glycine in the parental protein C7.GAT, and because 5′ thymine was not excluded by the two helices mentioned above.

Asparagine was also frequently selected in position 6. Helix HRT-T-LTN (SEQ ID NO: 94) and RSD-T-LSN (SEQ ID NO: 74) displayed excellent specificity for 5′ adenine. However, Asn⁶ also seemed to impart specificity for both adenine and guanine, suggesting an interaction with the N7 common to both nucleotides. Computer modeling of the helix binding to 5′-AGG-3′ (RSD-H-LTN (SEQ ID NO: 71)), based on the coordinates of finger 2, binding to 5′-TGG-3′, in the Zif268/DNA crystal structure (RSD-H-LTT(SEQ ID NO: 62); [Elrod-Erickson et al., (1996) Structure 4(10), 1171-1180]), suggested that the Nd of Asn⁶ would be approximately 4.5 Å from N7 of the 5′ adenine. A modest reorientation of the α-helix which is considered within the range of canonical docking orientations [Pabo et al., (2000) J. Mol. Biol. 301, 597-624], could plausibly bring the Nd within hydrogen bonding distance, analogous to the reorientation observed when glutamate rather than arginine appears in position −1. However, it is interesting to speculate why Asn⁶ was selected in this 5′-ANN-3′ recognition set while the longer Gln⁶ was not. Gln⁶, being more flexible, may have been able to stabilize other interactions that were selected against during phage display. Alternatively, the shorter side chain of Asn⁶ might accommodate an ordered water molecule that could contact the 5′ nucleotide without reorientation of the helix.

The final residue to be considered is Arg⁶. It was somewhat surprising that Arg⁶ was selected so frequently on 5′-ANN-3′ targets because in our previous studies, it was unanimously selected to recognize a 5′ guanine with high specificity [Segal et al., (1999) Proc Natl Acad Sci USA 96(6), 2758-2763]. However, in the previous study, Arg⁶ primarily specified 5′ adenine, in some cases in addition to recognition of a 5′ guanine. Computer modeling of helix binding to 5′-ACA-3¹′ (SPA-D-LTR (SEQ ID NO: 95)), based on the coordinates of finger 1 QSG-S-LTR (SEQ ID NO: 91) of a Zif268 variant binding 5′-GCA-3′ [Elrod-Erickson et al., (1998) Structure 6(4), 451-464], suggested that Arg⁶ could easily adopt a configuration that allowed it to make a cross-strand hydrogen bond to O4 of a thymine base-paired to 5′ adenine. In fact, Arg⁶ could bind with good geometry to both the O4 of thymine and O6 of a guanine base-paired to a middle cytosine. Such an interaction is consistent with the fact that Arg⁶ was selected almost unanimously when the target sequence was 5′-ACN-3′. The expectation for arginine to facilitate multiple interactions is compelling. Several lysines in TFIIIA were observed by NMR to be conformationally flexible [Foster et al., (1997) Nat. Struct. Biol. 4(8), 605-608], and Gln⁻¹ behaves in a manner which suggests flexibility [Dreier et al., (2000) J. Mol. Biol. 303, 489-5021. Arginine has more rotatable bonds and more hydrogen bonding potential than lysine or glutamine and it is attractive to speculate that Arg⁶ is not limited to recognition of 5′ guanine.

Amino acid residues in positions −1 and 3 were generally selected in analogy to their 5′-GNN-3′ counterparts with two exceptions. His⁻¹ was selected for pAGT and pATT, recognizing a 3′ thymine, and Ser⁻¹ for pACA, recognizing a 3′ adenine. While Gln³ was frequently used to specify a 3′ adenine in subsites of the type 5′-GNN-3′, a new element of 3′ adenine recognition was suggested from this study involving Ser⁻¹ selected for domains recognizing the 5′-ACA-3′ subsite which can make a hydrogen bond with the 3′ adenine. Computer modeling demonstrates that Ala², co-selected in the helix SPA-D-LTR (SEQ ID NO: 95), can potentially make a van der Waals contact with the methyl group of the thymine base-paired to 3′ adenine. The best evidence that Ala² might be involved is that helix SPA-D-LTR (SEQ ID NO: 95) is strongly specific for 3′ adenine while SHS-D-LVR, (SEQ ID NO-96) is not. Gln⁻¹ is often sufficient for 3′ adenine recognition. However, data from our previous studies suggested that the side chain of Gln⁻¹ can adopt multiple conformations, enabling, for example, recognition of 3′ thymine [Nardelli et al., (1992) Nucleic Acids Res. 20(16), 4137-44; Elrod-Erickson et al., (1998) Structure 6(4), 451-464; Dreier et al., (2000) J. Mol. Biol. 303, 489-502]. Ala² in combination with Ser⁻¹ may be an alternative means to specificity a 3′ adenine.

Another interaction not observed in the 5′-GNN-3′ study is the cooperative recognition of 3′ thymine by His⁻¹ and the residue at position 2. In finger 1 of the crystal structure of the Tramtrak/DNA complex, helix HIS-N-FCR (SEQ ID NO: 97) binds the subsite 5′-GAT-3′ [Fairall et al., (1993) Nature (London) 366(6454), 483-7]. The His⁻¹ ring is perpendicular to the plane of the 3′ thymine base and is approximately 4 Å from the methyl group. Ser2 additionally makes a hydrogen bond with O4 of 3′ thymine. A similar set of contacts can be envisioned by computer modeling for the recognition of 5′-ATT-3 by helix HKN-A-LQN (SEQ ID NO: 98). Asn² in this helix has the potential not only to hydrogen bond with 3′ thymine but also with the adenine base-paired to thymine. His⁻¹ was also found for the helix binding 5′-AGT-3′ (HRT-T-LLN (SEQ ID NO: 99)) in combination with a Thr². Thr is structurally similar to Ser and might be involved in a similar recognition mechanism.

In conclusion, the results of the characterization of zinc finger domains reported in this study binding 5′-ANN-3′ DNA subsites is consistent with the overall view that there is no general recognition code, which makes rational design of additional domains difficult. However, phage display selections can be applied and pre-defined zinc finger domains can serve as modules for the construction of artificial transcription factors. The domains characterized here enables targeting of DNA sequences other than 5′-(GNN)₆-3′. This is an important supplement to existing domains, since G/C-rich sequences often contain binding sites for cellular proteins and 5′(GNN)₆-3′ sequences may not be found in all promoters.

One conclusion that can be drawn is that a variety of amino acid residues at position 6 of the heptapeptide can specify an adenine at the 5′-position of the triplet subsite. These residues include alanine (A), arginine (R), asparagine (N), aspartate (D), glutamine (Q), glutamate (E), threonine (T), and valine (V).

Accordingly, in view of these results, rational design was performed to develop additional zinc fingers that bound the 5′-(AGC)-3′ subsite with a substantial degree of affinity and specificity. This was done by studying the binding profiles of many mutant proteins and made mutations based on proteins that seemed to have favorable interactions with the 5′-(AGC)-3′ subsite as a target sequence. Site-directed mutagenesis was carried out as described in Example 2, below, to develop these additional zinc fingers. The fingers developed by this strategy include: DPG-A-LIN (SEQ ID NO: 1), ERS-H-LRE (SEQ ID NO: 2); and DPG-H-LTE (SEQ ID NO-3).

Notwithstanding the lack of a general recognition code, these results provide a number of guidelines for the determination of sequences within the present invention to one of ordinary skill in the art. Some of these guidelines are also useful for selection of zinc finger domains specifically binding sequences of the form 5′-(AGC)-3′. These guidelines include the following: (1) For subsites containing a 3′-cytosine, Gln, Asn, Ser, Gly, His, or Asp are typically preferred in position −1. (2) For the target site 5′-AGN-3′, His is preferred at position 3. (3) For the target site 5′-AGC-3′ Trp and Thr are typically preferred at position 3; His is also possible. (4) Positions 1, 2, and 5 can vary widely. These are only guidelines, and the secondary or tertiary structure of a protein or polypeptide incorporating a zinc finger domain according to the present invention can lead to different amino acids being preferred for recognition of particular subsites or particular nucleotides at a defined position of such subsites. Additionally, the conformation of a particular zinc finger moiety within a protein having a plurality of zinc finger moieties can affect the binding.

Other amino acid residues are also subject to mutation or substitution. For example, leucine is often located in position 4 of the seven-amino acid domain and packs into the hydrophobic core of the protein. Accordingly, the leucine in position 4 can be replaced with other relatively small hydrophobic residues, such as valine and isoleucine, without disturbing the three-dimensional structure or function of the protein. Alternatively, the leucine in position 4 can also be replaced with other hydrophobic residues such as phenylalanine or tryptophan.

Other amino acid substitutions are possible. When G is in the middle position of the triplet, His is a possibility for position 3 of the helix and can replace another amino acid there. When the last two bases of the triplet are GC, Trp and Thr are alternatives at position 3 and can replace another amino acid there. Cys is also an alternative for position 4, particularly when Leu was present there.

The following table (Table 2) describes a potentially useful range of amino acid substitutions assuming that the 5′-base is A, as would be the case in the triplet 5′-(AGC)-3′.

TABLE 2

| Middle | 3′ | Zinc Finger Amino | Amino Acid |
|---|---|---|---|
| Base | Base | Acid Position | Alternatives |
| A | A | −1 | Q, N, S |
| C | A | −1 | S |
| N | G | −1 | R, N, Q, H, S, T, I |
| N | G | 2 | D |
| N | T | −1 | R, N, Q, H, S, T, A, C |
| N | C | −1 | Q, N, S, G, H, D |
| A | N | 3 | H, N, G, V, P, I, K |
| C | N | 3 | T, D, H, K, R, N |
| C | C | 3 | N, H, S, D, T, Q, G |
| C | G | 3 | T, H, S, D, N, Q, G |
| G | N | 3 | H |
| G | G/T | 3 | S, D, T, N, Q, G, H |
| G | C | 3 | W, T, H |
| G | N | 3 | H |
| T | A/G | 3 | S, A |
| T | C/T | 3 | H |
| N | A | −1 | R |
| N | T | −1 | S, T, H |
| N | N | 4 | L, V, I, C |

In Table 2, particularly preferred amino acids are underlined. “N” is any of the four possible naturally-occurring nucleotides (A, C, G, or T).

Additionally, inspection of the domains binding nucleotide sequences of the form 5′-(AGC)-3′ reveals that residues 4, 5, and 6 can be selected from LIN, LRE, and LTE, and that these three-amino-acid partial sequences can be interchanged when the 3′-residue of the nucleic acid subsite to be recognized is A. This finding can be used to generate additional zinc finger domains.

Accordingly, preferred zinc finger domains included in polypeptides according to the present invention and binding sequences of the form 5′-(AGC)-3′ include the following: SEQ ID NO: 1 through SEQ ID NO: 57.

Of these, SEQ ID NO: 1 through SEQ ID NO: 10 are particularly preferred; SEQ ID NO: 1, SEQ ID NO: 2, and SEQ ID NO: 3 are more particularly preferred.

SEQ ID NO: 4 through SEQ ID NO: 57 are derived from the sequences of SEQ ID NO: 1, SEQ ID NO: 2, or SEQ ID NO: 3 by the rules of general applicability for substitution of amino acids set forth above in Tables 1 and 2 or by the interchangeability of the partial motifs LIN, LRE, and LTE at positions 4, 5, and 6, respectively, of these domains. SEQ ID NO: 4 through SEQ ID NO: 10 are derived by the rules set forth in Table 1. SEQ ID NO: 11 through SEQ ID NO: 26 are derived by the rules set forth in Table 2. SEQ ID NO: 27 through SEQ ID NO: 57 are derived by the interchangeability of the partial motifs LIN, LRE, and LTE at positions 4, 5, and 6, respectively, of these domains. Accordingly, these sequences are within the scope of the invention and polypeptides incorporating these sequences and binding nucleotide subsites of the form 5′-(AGC)-3′ are also within the scope of the invention. These sequences are:

DPG-A-LIN (SEQ ID NO: 1)
ERS-H-LRE (SEQ ID NO: 2)
DPG-H-LTE (SEQ ID NO: 3)
EPG-A-LIN (SEQ ID NO: 4)
DRS-H-LRE (SEQ ID NO: 5)
EPG-H-LTE (SEQ ID NO: 6)
ERS-L-LRE (SEQ ID NO: 7)
DRS-K-LRE (SEQ ID NO: 8)
DPG-K-LTE (SEQ ID NO: 9)
EPG-K-LTE (SEQ ID NO: 10)
DPG-W-LIN (SEQ ID NO: 11)
DPG-T-LIN (SEQ ID NO: 12)
DPG-H-LIN (SEQ ID NO: 13)
ERS-W-LIN (SEQ ID NO: 14)
ERS-T-LIN (SEQ ID NO: 15)
DPG-W-LTE (SEQ ID NO: 16)
DPG-T-LTE (SEQ ID NO: 17)
EPG-W-LIN (SEQ ID NO: 18)
EPG-T-LIN (SEQ ID NO: 19)
EPG-H-LIN (SEQ ID NO: 20)
DRS-W-LRE (SEQ ID NO: 21)
DRS-T-LRE (SEQ ID NO: 22)
EPG-W-LTE (SEQ ID NO: 23)
EPG-T-LTE (SEQ ID NO: 24)
ERS-W-LRE (SEQ ID NO: 25)
ERS-T-LRE (SEQ ID NO: 26)
DPG-A-LRE (SEQ ID NO: 27)
DPG-A-LTE (SEQ ID NO: 28)
ERS-H-LIN (SEQ ID NO: 29)
ERS-H-LTE (SEQ ID NO: 30)
DPG-H-LIN (SEQ ID NO: 31)
DPG-H-LRE (SEQ ID NO: 32)
EPG-A-LRE (SEQ ID NO: 33)
EPG-A-LTE (SEQ ID NO: 34)
DRS-H-LIN (SEQ ID NO: 35)
DRS-H-LTE (SEQ ID NO: 36)
EPG-H-LRE (SEQ ID NO: 37)
ERS-K-LIN (SEQ ID NO: 38)
ERS-K-LTE (SEQ ID NO: 39)
DRS-K-LIN (SEQ ID NO: 40)
DRS-K-LTE (SEQ ID NO: 41)
DPG-K-LIN (SEQ ID NO: 42)
DPG-K-LRE (SEQ ID NO: 43)
EPG-K-LIN (SEQ ID NO: 44)
EPG-K-LRE (SEQ ID NO: 45)
DPG-W-LRE (SEQ ID NO: 46)
DPG-T-LRE (SEQ ID NO: 47)
DPG-H-LRE (SEQ ID NO: 48)
DPG-H-LTE (SEQ ID NO: 49)
ERS-W-LTE (SEQ ID NO: 50)
ERS-T-LTE (SEQ ID NO: 51)
EPG-W-LRE (SEQ ID NO: 52)
EPG-T-LRE (SEQ ID NO: 53)
DRS-W-LIN (SEQ ID NO: 54)
DRS-W-LTE (SEQ ID NO: 55)
DRS-T-LIN (SEQ ID NO: 55)
DRS-T-LTE (SEQ ID NO: 57)

In one embodiment, a polypeptide of the invention contains a binding region that has an amino acid sequence with the same nucleotide binding characteristics as any of SEQ ID NO: 1 through SEQ ID NO: 57. A detailed description of how those binding characteristics were determined can be found hereinafter in the Examples. Such a polypeptide competes for binding to a nucleotide target with any of SEQ ID NO: 1 through SEQ ID NO: 57. That is, a preferred polypeptide contains a binding region that will displace, in a competitive manner, the binding of any of SEQ ID NO: 1 through SEQ ID NO: 57. Means for determining competitive binding are well known in the art. More preferably, the polypeptide contains a binding region that has an amino acid sequence with the same nucleotide binding characteristics as any of SEQ ID NO: 1 through SEQ ID NO: 10, competes for binding to a nucleotide target with any of SEQ ID NO: 1 through SEQ ID NO: 10, or contains a binding region that will displace, in a competitive manner, the binding of any of SEQ ID NO: 1 through SEQ ID NO: 10. Still more preferably, the polypeptide contains a binding region that has an amino acid sequence with the same nucleotide binding characteristics as any of SEQ ID NO: 1, SEQ ID NO: 2, or SEQ ID NO: 3, competes for binding to a nucleotide target with any of SEQ ID NO: 1, SEQ ID NO: 2, or SEQ ID NO: 3, or contains a binding region that will displace, in a competitive manner, the binding of any of SEQ ID NO: 1, SEQ ID NO: 2, or SEQ ID NO: 3. Preferably, the binding region has the amino acid sequence of any of SEQ ID NO: 1 through SEQ ID NO: 57. More preferably, the binding region has the amino acid sequence of any of SEQ ID NO: 1 through SEQ ID NO: 10. Still more preferably, the binding region has the amino acid sequence of any of SEQ ID NO: 1, SEQ ID NO: 2, or SEQ ID NO: 3.

Also within the scope of the present invention are polypeptides that differ from the polypeptides disclosed above, such as polypeptides including therein any of SEQ ID NO: 1 through SEQ ID NO: 57, any of SEQ ID NO: 1 through SEQ ID NO: 10, or any of SEQ ID NO: 1, SEQ ID NO: 2, or SEQ ID NO: 3 by no more than two conservative amino acid substitutions and that have a binding affinity for the desired subsite or target region of at least 80% as great as the polypeptide before the substitutions are made. In terms of dissociation constants, this is equivalent to a dissociation constant no greater than 125% of that of the polypeptide before the substitutions are made. In this context, the term “conservative amino acid substitution” is defined as one of the following substitutions: Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. Preferably, the polypeptide differs from the polypeptides described above by no more than one conservative amino acid substitution.

Additionally, proteins or polypeptides incorporating zinc fingers can be molecularly modeled, as detailed below in Example 11. One suitable computer program for molecular modeling is Insight II. Molecular modeling can be used to generate other zinc finger moieties based on variations of zinc finger moieties described herein and that are within the scope of the invention. When modeling establishes that such variations have a hydrogen-bonding pattern that is substantially similar to that of a zinc finger moiety within the scope of the invention and that has been used as the basis for modeling, such variations are also within the scope of the invention. As used herein, the term “substantially similar” with respect to hydrogen bonding pattern means that the same number of hydrogen bonds are present, that the bond angle of each hydrogen bond varies by no more than about 10 degrees, and that the bond length of each hydrogen bond varies by no more than about 0.2 Å.

Typically, binding between the polypeptide and the DNA of appropriate sequence occurs with a K_{D} of from 1 μM to 10 μM. Preferably binding occurs with a K_{D} of from 10 μM to 1 μM, from 10 pM to 100 nM, from 100 pM to 10 nM and, more preferably with a K_{D} of from 1 nM to 10 nM. These binding parameters also characterize binding of other polypeptides incorporating these polypeptides, such as the polypeptide compositions described below herein.

Accordingly, other zinc finger nucleotide binding domains can be included in polypeptides according to the present invention. All of these domains include a 7-amino acid zinc finger domain wherein the seven amino acids of the domain are numbered from −1 to 6. These domains include: (1) a zinc finger nucleotide binding domain specifically binding the nucleotide sequence 5′-(AGC)-3′, wherein the amino acid residue of the domain numbered −1 is selected from the group consisting of Q, N, S, G, H, and D; (2) a zinc finger nucleotide binding domain specifically binding the nucleotide sequence 5′-(AGC)-3′, wherein the amino acid residue of the domain numbered 3 is selected from the group consisting of W, T, and H; (3) a zinc finger nucleotide binding domain specifically binding the nucleotide sequence 5′-(AGC)-3′ wherein the amino acid residue of the domain numbered 4 is selected from the group consisting of L, V, I, and C; (4) a zinc finger nucleotide binding domain specifically binding the nucleotide sequence 5′-(AGC)-3′ wherein the amino acid residue of the domain numbered 6 is selected from the group consisting of A, R, N, D, Q, E, T, and V; and (5) a zinc finger nucleotide binding domain specifically binding the nucleotide sequence 5′-(AGC)-3′ wherein the amino acid residue of the domain numbered −1 is selected from the group consisting of D and E and wherein the residues of the domain numbering 4 through 6 are selected from the group consisting of LIN, LRE, and LTE.

Still other zinc finger nucleotide binding domains that can be incorporated in polypeptides according to the present invention can be derived from the domains described above, namely SEQ ID NO: 1 through SEQ ID NO: 57, by site-derived mutagenesis and screening. Site-directed mutagenesis techniques, also known as site-specific mutagenesis techniques are well known in the art and need not be described in detail here. Such techniques are described, for example, in J. Sambrook & D. W. Russell, “Molecular Cloning; A Laboratory Manual” (3^{rd} ed., Cold Spring Harbor Laboratory Press, Cold Spring Harbor, N.Y., 2001), v.2, ch. 13, pp. 13.1-13.56.

III. Polypeptide Compositions

In another aspect, the present invention provides a polypeptide composition that comprises a plurality of zinc finger-nucleotide binding domains operatively linked in such a manner to specifically bind a nucleotide target motif defined as 5′-(AGC)ₙ-3′, where n is an integer greater than 1. The target motif can be located within any longer nucleotide sequence (e.g., from 3 to 13 or more TNN, CNN, GNN, ANN or NNN sequences). Preferably, n is an integer from 2 to 18, more preferably from 2 to 12, and still more preferably from 2 to 6. The individual polypeptides are preferably linked with oligopeptide linkers. Such linkers preferably resemble a linker found in naturally occurring zinc finger proteins. A preferred linker for use in the present invention is the amino acid residue sequence TGEKP (SEQ ID NO: 100). Modifications of this linker can also be used. For example, the glutamic acid (E) at position 3 of the linker can be replaced with aspartic acid (D). The threonine (T) at position 1 can be replaced with serine(S). The glycine (G) at position 2 can be replaced with alanine (A). The lysine (K) at position 4 can be replaced with arginine (R). Another preferred linker for use in the present invention is the amino acid residue sequence TGGGGSGGGGTGEKP (SEQ ID NO: 101). This longer linker can be used when it is desired to have the two halves of a longer plurality of zinc finger binding polypeptides operate in a substantially independent manner. Modifications of this longer linker can also be used. For example, the polyglycine runs of four glycine (G) residues each can be of greater or lesser length (i.e., 3 or 5 glycine residues each). The serine residue (S) between the polyglycine runs can be replaced with threonine (T). The TGEKP (SEQ ID NO: 100) moiety that comprises part of the linker TGGGGSGGGGTGEKP (SEQ ID NO: 101) can be modified as described above for the TGEKP (SEQ ID NO: 100) linker alone. Other linkers such as glycine or serine repeats are well known in the art to link peptides (e.g., single chain antibody domains) and can be used in a composition of this invention. The use of a linker is not required for all purposes and can optionally be omitted.

Other linkers are known in the art and can alternatively be used. These include the linkers LRQKDGGGSERP (SEQ ID NO: 102), LRQKDGERP (SEQ ID NO.: 103), GGRGRGRGRQ (SEQ ID NO: 104), QNKKGGSGDGKKKQHI (SEQ ID NO: 105), TGGERP (SEQ ID NO: 106), ATGEKP (SEQ ID NO: 107), and GGGSGGGGEGP (SEQ ID NO: 116), as well as derivatives of those linkers in which amino acid substitutions are made as described above for TGEKP (SEQ ID NO: 100) and TGGGGSGGGGTGEKP(SEQ ID NO: 101). For example, in these linkers, the serine (S) residue between the diglycine or polyglycine runs in QNKKGGSGDGKKKQHI (SEQ ID NO: 105) or GGGSGGGGEGP (SEQ ID NO: 116) can be replaced with threonine (T). In GGGSGGGGEGP (SEQ ID NO: 116), the glutamic acid (E) at position 9 can be replaced with aspartic acid (D). Polypeptide compositions including these linkers and derivatives of these linkers are included in polypeptide compositions of the present invention

In these polypeptide compositions, each of the zinc finger domains is of the sequence SEQ ID NO: 1 to SEQ ID NO: 57. Typically, each of the zinc finger domains is of the sequence SEQ ID NO: 1 to SEQ ID NO: 10 Preferably, each of the zinc finger domains is of the sequence SEQ ID NO: 1, SEQ ID NO: 2, or SEQ ID NO: 3.

Alternatively, in these polypeptide compositions, each of these zinc finger domains contains a binding region that has an amino acid sequence with the same nucleotide binding characteristics-as any of SEQ ID NO: 1 through SEQ ID NO: 57, that competes for binding to a nucleotide target with any of SEQ ID NO: 1 through SEQ ID NO: 57, or that will displace, in a competitive manner, the binding of any of SEQ ID NO: 1 through SEQ ID NO: 57. In this alternative, preferably, each of these zinc finger domains contains a binding region that has an amino acid sequence with the same nucleotide binding characteristics as any of SEQ ID NO: 1 through SEQ ID NO: 10, that competes for binding to a nucleotide target with any of SEQ ID NO: 1 through SEQ ID NO: 10, or that will displace, in a competitive manner, the binding of any of SEQ ID NO: 1 through SEQ ID NO: 10. More preferably, each of these zinc finger domains contains a binding region that has an amino acid sequence with the same nucleotide binding characteristics as any of SEQ ID NO: 1, SEQ ID NO: 2, or SEQ ID NO: 3, that competes for binding to a nucleotide target with any of SEQ ID NO: 1, SEQ ID NO: 2, or SEQ ID NO: 3, or that will displace, in a competitive manner, the binding of any of SEQ ID NO: 1, SEQ ID NO: 2, or SEQ ID NO: 3.

In another alternative, each of these zinc finger domains contains a binding region that differs from the binding region disclosed above, such as binding regions including therein any of SEQ ID NO: 1 through SEQ ID NO: 57, any of SEQ ID NO: 1 through SEQ ID NO: 10, or any of SEQ ID NO: 1, SEQ ID NO: 2, or SEQ ID NO: 3 by no more than two conservative amino acid substitutions and that have a binding affinity for the desired subsite or target region of at least 80% as great as the binding region before the substitutions are made. In assessing the binding affinity for the desired subsite or target region in these multi-binding region polypeptides, the binding affinity is determined in the absence of interference from other binding regions.

In yet another alternative, in polypeptide compositions according to the present invention as described above, each of the zinc finger domains is a domain such as the following: (1) a zinc finger nucleotide binding domain specifically binding the nucleotide sequence 5′-(AGC)-3′, wherein N is any of A, C, G, or T, wherein the amino acid residue of the domain numbered −1 is selected from the group consisting of Q, N, S, G, H, and D; (2) a zinc finger nucleotide binding domain specifically binding the nucleotide sequence 5′-(AGC)-3′, wherein the amino acid residue of the domain numbered 3 is selected from the group consisting of W, T, and H; and (3) a zinc finger nucleotide binding domain specifically binding the nucleotide sequence 5′-(AGC)-3′ wherein the amino acid residue of the domain numbered 4 is selected from the group consisting of L, V, I, and C.

In still other alternatives, any of the zinc finger nucleotide binding domains described above can be included in a polypeptide composition according to the present invention.

Other alternatives for the binding regions of these polypeptides, including binding regions generated by molecular modeling as described above, are within the scope of the invention.

In still anotheralternative, the polypeptide composition can comprise a bispecific zinc finger protein comprising two halves, each half comprising six zinc finger nucleotide binding domains, where at least one of the halves includes at least one domain binding a target nucleotide sequence of the form, 5′-(AGC)-3′, such that the two halves of the bispecific zinc fingers can operate independently. The two halves can be linked by a linker such as the amino acid residue sequence TGGGGSGGGGTGEKP (SEQ ID NO: 101) or another linker as described above. Typically, the linker in this form of bispecific zinc finger protein will include from about 12 to about 18 amino acid residues.

In another alternative, the polypeptide compositions can include, in addition to the binding regions that specifically bind nucleotide subsites or target regions with the sequence 5′-(AGC)-3′, one or more polypeptides that include binding regions that specifically bind nucleotide subsites or target regions with the sequence 5′-(ANN)-3′, 5′-(CNN)-3′, 5′-(GNN)-3′, or 5′-(TNN)-3′. Binding regions that specifically bind nucleotide subsites with the sequence 5′-(ANN)-3′ are disclosed, for example, in U.S. Patent Application Publication No. 2002/0165356 by Barbas et al., incorporated herein by this reference. Binding regions that specifically bind nucleotide subsites with the sequence 5′-(CNN)-3′ are disclosed, for example, in U.S. Patent Application Publication No. 2004/0224385 by Barbas et al., incorporated herein by this reference. Binding regions that specifically bind nucleotide subsites with the sequence 5′-(GNN)-3′ are disclosed, for example, in U.S. Pat. No. 6,610,512 to Barbas and in U.S. Pat. No. 6,140,081 to Barbas, both incorporated herein by this reference.

If the polypeptide includes binding regions that specifically bind nucleotide subsites of the structure 5′-(ANN)-3′, 5′-(CNN)-3′, 5′-(TNN)-3′, or 5′-(TNN)-3′, they can be in any order within the polypeptide, as long as the polypeptide has at least one binding region that binds a nucleotide subsite of the structure 5′-(ACG)-3′. For example, but not by way of limitation, the polypeptide can include a block of binding regions, all of which bind nucleotide subsites of the structure 5′-(ACG)-3′, or have binding regions binding nucleotide subsites of the structure 5′-(ACG)-3′ interspersed with binding regions binding nucleotide subsites of the structure 5′-(ANN)-3′, 5′-(CNN)-3′, 5′-(GNN)-3′, or 5′-(TNN)-3′. The polypeptide can include 1, 2, 3, 4, 5, 6, 7, 8, 9, 10, 11, 12, 13, 14, 15, 16, 17, 18, or more binding regions, each binding a subsite of the structure 5′-(ANN)-3′, 5′-(CNN)-3′, 5′-(GNN)-3′, or 5′-(TNN)-3′, again as long as the polypeptide has at least one binding region that binds a nucleotide subsite of the structure 5′-(AGC)-3′. In one alternative, all of the binding regions within the polypeptide bind nucleotide subsites of the structure 5′-(ACG)-3′.

A polypeptide composition of this invention can be operatively linked to one or more functional polypeptides. Such functional polypeptides can be the complete sequence of proteins with a defined function, or can be derived from single or multiple domains that occur within a protein with a defined function. Such functional polypeptides are well known in the art and can be a transcription regulating factor such as a repressor or activation domain or a polypeptide having other functions. Exemplary and preferred functional polypeptides that can be incorporated are nucleases, lactamases, integrases, methylases, nuclear localization domains, and restriction enzymes such as endo- or exonucleases, as well as other domains with enzymatic activity such as hydrolytic activity (See, e.g. Chandrasegaran and Smith, Biol. Chem., 380:841-848, 1999). Typically, the operative linkage occurs by creating a single polypeptide joining the zinc finger domains with the other functional polypeptide or polypeptides to form a fusion protein, the linkage can occur directly or through one or more linkers as described above. Among the other polypeptides that can be joined to a polypeptide composition according to the present invention, for example, are the nuclease catalytic domain of Fokl to generate a construct that can direct site-specific cleavage at a chosen genomic target.

An exemplary repression domain polypeptide is the ERF repressor domain (ERD) (Sgouras, D. N., Athanasiou, M. A., Beal, G. J., Jr., Fisher, R. J., Blair, D. G. & Mavrothalassitis, G. J. (1995) EMBO J. 14, 4781-4793), defined by amino acids 473 to 530 of the ets2 repressor factor (ERF). This domain mediates the antagonistic effect of ERF on the activity of transcription factors of the ets family. A synthetic repressor is constructed by fusion of this domain to the N- or C-terminus of the zinc finger protein. A second repressor protein is prepared using the Krüppel-associated. box (KRAB) domain (Margolin, J. F., Friedman, J. R., Meyer, W., K.-H., Vissing, H., Thiesen, H.-J. & Rauscher III, F. J. (1994) Proc. Natl. Acad. Sci. USA 91, 4509-4513). This repressor domain is commonly found at the N-terminus of zinc finger proteins and presumably exerts its repressive activity on TATA-dependent transcription in a distance-and orientation-independent manner (Pengue, G. & Lania, L. (1996) Proc. Natl. Acad. Sci. USA 93, 1015-1020), by interacting with the RING finger protein KAP-1 (Friedman, J. R., Fredericks, W. J., Jensen, D. E., Speicher, D. W., Huang, X.-P., Neilson, E. G. & Rauscher III, F. J. (1996) Genes & Dev. 10, 2067-2078). We utilized the KRAB domain found between amino acids 1 and 97 of the zinc finger protein KOX1 (Margolin, J. F., Friedman, J. R., Meyer, W., K.-H., Vissing, H., Thiesen, H.-J. & Rauscher III, F. J3 (1994) Proc. Natl. Acad. Sci. USA 91, 4509-4513). In this case an N-terminal fusion with a zinc-finger polypeptide is constructed. Finally, to explore the utility of histone deacetylation for repression, amino acids 1 to 36 of the Mad mSIN3 interaction domain (SID) are fused to the N-terminus of the zinc finger protein (Ayer, D. E., Laherty, C. D., Lawrence, Q. A., Armstrong, A. P. & Eisenman, R. N. (1996) Mol. Cell. Biol. 16, 5772-5781). This small domain is found at the N-terminus of the transcription factor Mad and is responsible for mediating its transcriptional repression by interacting with mSIN3, which in turn interacts the co-repressor N-COR and with the histone deacetylase mRPD1 (Heinzel, T., Lavinsky, R. M., Mullen, T.-M., Soderstrom, M., Laherty, C. D., Torchia, J., Yang, W.-M., Brard, G., & Ngo, S. D. (1997) Nature 387,43-46). To examine gene-specific activation, transcriptional activators are generated by fusing the zinc finger polypeptide to amino acids 413 to 489 of the herpes simplex virus VP16 protein (Sadowski, I., Ma, J., Triezenberg, S. & Ptashne, M. (1988) Nature 335, 563-564), or to an artificial tetrameric repeat of VP16's minimal activation domain (Seipel, K., Georgiev, O. & Schaffler, W. (1992) EMBO J. 11, 4961-4968), termed VP64.

A polypeptide of this invention as set forth above can be operatively linked to one or more transcription modulating or regulating factors. Modulating factors such as transcription activators or transcription suppressors or repressors are well known in the art. Means for operatively linking polypeptides to such factors are also well known in the art. Exemplary and preferred such factors and their use to modulate gene expression are discussed in detail hereinafter.

In order to test the concept of using zinc finger proteins as gene-specific transcriptional regulators, six-finger proteins are fused to a number of effector domains. Transcriptional repressors are generated by attaching either of three human-derived repressor domains to the zinc finger protein. The first repressor protein is prepared using the ERF repressor domain (ERD) (Sgouras, D. N., Athanasiou, M. A., Beal, G. J., Jr., Fisher, R. J., Blair, D. G. & Mavrothalassitis, G. J. (1995) EMBO J. 14, 4781-4793), defined by amino acids 473 to 530 of the ets2 repressor factor (ERF). This domain mediates the antagonistic effect of ERF on the activity of transcription factors of the ets family. A synthetic repressor is constructed by fusion of this domain to the C-terminus of the zinc finger protein. The second repressor protein is prepared using the Krüppel-associated box (KRAB) domain (Margolin, J. F., Friedman, J. R., Meyer, W., K.-H., Vissing, H., Thiesen, H.-J. & Rauscher III F. J. (1994) Proc. Natl. Acad. Sci. USA 91, 4509-4513). This repressor domain is commonly found at the N-terminus of zinc finger proteins and presumably exerts its repressive activity on TATA-dependent transcription in a distance- and orientation-independent manner (Pengue, G. & Lania, L. (1996) Proc. Natl. Acad. Sci. USA 93, 1015-1020), by interacting with the RING finger protein KAP-1 (Friedman, J. R., Fredericks, W. J., Jensen, D. E., Speicher, D. W., Huang, X.-P., Neilson, E. G. & Rauscher III, F. J. (1996) Genes & Dev. 10, 2067-2078). We utilize the KRAB domain found between amino acids 1 and 97 of the zinc finger protein KOX1 (Margolin, J. F., Friedman, J. R., Meyer, W., K.-H., Vissing, H., Thiesen, H.-J. & Rauscher III, F. J. (1994) Proc. Natl. Acad. Sci. USA 91, 4509-4513). In this case an N-terminal fusion with the six-finger protein is constructed. Finally, to explore the utility of histone deacetylation for repression, amino acids 1 to 36 of the Mad mSIN3 interaction domain (SID) are fused to the N-terminus of a zinc finger protein (Ayer, D. E., Laherty, C. D., Lawrence, Q. A., Armstrong, A. P. & Eisenman, R. N. (1996) Mol. Cell. Biol. 16, 5772-5781). This small domain is found at the N-terminus of the transcription factor Mad and is responsible for mediating its transcriptional repression by interacting with mSIN3, which in turn interacts the co-repressor N-CoR and with the histone deacetylase mRPD1 (Heinzel, T., Lavinsky, R. M., Mullen, T.-M., Soderstrom, M., Laherty, C. D., Torchia, J., Yang, W.-M., Brard, G., & Ngo, S. D. (1997) Nature 387, 43-46). Another alternative is direct fusion with a histone deacetylase such as HDAC1.

To examine gene-specific activation, transcriptional activators are generated by fusing the zinc finger protein to amino acids 413 to 489 of the herpes simplex virus VP 16 protein (Sadowski, I., Ma, J., Triezenberg, S. & Ptashne, M. (1988) Nature 335, 563-564), or to an artificial tetrameric repeat of VP16's minimal activation domain, DALDDFDLDML (SEQ ID NO: 108) (Seipel, K., Georgiev, O. & Schaffner, W. (1992.) EMBO J. 11, 49614968), termed VP64.

Reporter constructs containing fragments of the erbB-2 promoter coupled to a luciferase reporter gene are generated to test the specific activities of our designed transcriptional regulators. The target reporter plasmid contains nucleotides −758 to −1 with respect to the ATG initiation codon. Promoter fragments display similar activities when transfected transiently into HeLa cells, in agreement with previous observations (Hudson, L. G., Ertl, A. P. & Gill, G. N. (1990) J. Biol. Chem. 265,4389-4393). To test the effect of zinc finger-repressor domain fusion constructs on erbB-2 promoter activity, HeLa cells are transiently co-transfected with zinc finger expression vectors and the luciferase reporter constructs. Significant repression is observed with each construct. The utility of gene-specific polydactyl proteins to mediate activation of transcription is investigated using the same two reporter constructs.

The data herein show that zinc finger proteins capable of binding novel 9- and 18-bp DNA target sites, as well as DNA target sites of other lengths, can be rapidly prepared using pre-defined domains recognizing 5′-(AGC)-3′ sites, or, in addition, domains recognizing 5′-(ANN)-3′, 5′-(CNN)-3′, 5′-(GNN)-3′, or 5′-(TNN)-3′ sites as well as domains recognizing 5′-(AGC)-3′ sites. This information is sufficient for the preparation of 166 or 17 million novel six-finger proteins each capable of binding 18 bp of DNA sequence. This rapid methodology for the construction of novel zinc finger proteins has advantages over the sequential generation and selection of zinc finger domains proposed by others (Greisman, H. A. & Pabo, C. O. (1997) Science 275, 657-661) and takes advantage of structural information that suggests that the potential for the target overlap problem as defined above might be avoided in proteins targeting 5′-(AGC)-3′ sites. Using the complex and well studied erbB-2 promoter and live human cells, the data demonstrate that these proteins, when provided with the appropriate effector domain, can be used to provoke or activate expression and to produce graded levels of repression down to the level of the background in these experiments.

IV. Isolated Heptapeptides

Another aspect of the present invention is an isolated heptapeptide having an α-helical structure and that binds preferentially to a target nucleotide of the formula AGC. Preferred target nucleotides are as described above. The heptapeptides can be of sequences SEQ ID NO: 1 through SEQ ID NO: 57.

Preferably, the heptapeptide has the amino acid sequence of any of SEQ ID NO: 1 through SEQ ID NO: 10. More preferably, the heptapeptide has the amino acid sequence of SEQ ID NO: 1, SEQ ID NO: 2, or SEQ ID NO: 3.

In another alternative, a heptapeptide according to the present invention has an amino acid sequence with the same nucdeotide binding characteristics as any of SEQ ID NO: 1 through SEQ ID NO: 57. Such a heptapeptide competes for binding to a nucleotide target with any of SEQ ID NO: 1 through SEQ ID NO: 57. That is, the heptapeptide will displace, in a competitive manner, the binding of any of SEQ ID NO: 1 through SEQ ID NO: 57. More preferably, the heptapeptide has an amino acid sequence with the same nucleotide binding characteristics as any of SEQ ID NO: 1 through SEQ ID NO: 10, competes for binding to a nucleotide target with any of SEQ ID NO: 1 through SEQ ID NO: 10, or will displace, in a competitive manner, the binding of any of SEQ ID NO: 1 through SEQ ID NO: 10. Still more preferably, the heptapeptide has an amino acid sequence with the same nucleotide binding characteristics as any of SEQ ID NO: 1, SEQ ID NO: 2, or SEQ ID NO: 3, competes for binding to a nucleotide target with any of SEQ ID NO: 1, SEQ ID NO: 2, or SEQ ID NO: 3, or contains a binding region that will displace, in a competitive manner, the binding of any of SEQ ID NO: 1, SEQ ID NO: 2, or SEQ ID NO: 3.

In yet another alternative, the heptapeptide has an amino acid sequence selected from the group consisting of:

(1) the amino acid sequence of any, of SEQ ID NO: 1 through SEQ ID NO: 57; and

(2) an amino acid sequence differing from the amino acid sequence of any of SEQ ID NO: 1 through SEQ ID NO: 57 by no more than two conservative amino acid substitutions, wherein the dissociation constant is no greater than 125% of that of the polypeptide before the substitutions are made, and wherein a conservative amino acid substitution is one of the following substitutions: Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu.

In this alternative, preferably, the heptapeptide has an amino acid sequence selected from the group consisting of:

(1) the amino acid sequence of any of SEQ ID NO: 1 through SEQ ID NO: 10; and

(2) an amino acid sequence differing from the amino acid sequence of any of SEQ ID NO: 1 through SEQ ID NO: 10 by no more than two conservative amino acid substitutions, wherein the dissociation constant is no greater than 125% of that of the polypeptide before the substitutions are made, and wherein a conservative amino acid substitution is one of the following substitutions: Ala/Gly or Ser; Arg/Lys; Asn/Gln or His; Asp/Glu; Cys/Ser; Gln/Asn; Sly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; SerfThr; Thr/Ser; Trp/Tyr; Tyr/Trp or Phe; Val/Ile or Leu.

More preferably, in this alternative, the heptapeptide has an amino acid sequence selected from the group consisting of:

(1) the amino acid sequence of any of SEQ ID NO: 1, SEQ ID NO: 2, or SEQ ID NO: 3; and

(2) an amino acid sequence differing from the amino acid sequence of any of SEQ ID NO: 1, SEQ ID NO: 2, or SEQ ID NO: 3 by no more than two conservative amino acid substitutions, wherein the dissociation constant is no greater than 125% of that of the polypeptide before the substitutions are made, and wherein a conservative amino acid substitution is one of the following substitutions: Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Vat; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu.

In these alternatives, preferably the heptapeptide differs from the amino acid sequence of SEQ ID NO: 1 through SEQ ID NO: 57, SEQ ID NO: 1 through SEQ ID NO: 10, or SEQ ID NO: 1, SEQ ID NO: 2, or SEQ ID NO: 3 by no more than one conservative amino acid substitution.

In still another alternative, the heptapeptide is one of the following (wherein the residues of the heptapeptide are numbered from −1 to 6 as described above): (1) an isolated heptapeptide specifically binding the nucleotide sequence 5′-(ASC)-3′, wherein N is any of A, C, G, or T, wherein the amino acid residue of the domain numbered −1 is selected from the group consisting of Q, N, S, G, H, and D; (2) an isolated heptapeptide specifically binding the nucleotide sequence 5′-(AGC)-3′, wherein the amino acid residue of the domain numbered 3 is selected from the group consisting of W, T, and H; and (3) an isolated heptapeptide specifically binding the nucleotide sequence 5′-(AGC)-3′ wherein the amino acid residue of the domain numbered 4 is selected from the group consisting of L, V, I, and C.

V. Polynucleotides, Expression Vectors, and Transformed Cells

The invention includes a nucleotide sequence encoding a zinc finger-nucleotide binding peptide or polypeptide, including polypeptides, polypeptide compositions, and isolated heptapeptides as described above. DNA sequences encoding the zinc finger-nucleotide binding polypeptides of the invention, including native, truncated, and extended polypeptides, can be obtained by several methods. For example, the DNA can be isolated using hybridization procedures that are well known in the art. These include, but are not limited to: (1) hybridization of probes to genomic or cDNA libraries to detect shared nucleotide sequences; (2) antibody screening of expression libraries to detect shared structural features; and (3) synthesis by the polymerase chain reaction (PCR). RNA sequences of the invention can be obtained by methods known in the art (See, for example, Current Protocols in Molecular Biology, Ausubel, et al., Eds., 1989).

The development of specific DNA sequences encoding zinc finger-nucleotide binding polypeptides of the invention can be obtained by: (1) isolation of a double-stranded DNA sequence from the genomic DNA; (2) chemical manufacture of a DNA sequence to provide the necessary codons for the polypeptide of interest; and (3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA. Of these three methods for developing specific DNA sequences for use in recombinant procedures, the isolation of genomic DNA is the least common. This is especially true when it is desirable to obtain the microbial expression of mammalian polypeptides due to the presence of introns. For obtaining zinc finger derived-DNA binding polypeptides, the synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the formation of cDNA sequences. Among the standard procedures for isolating cDNA sequences of interest is the formation of plasmid-carrying cDNA libraries which are derived from reverse transcription of mRNA which is abundant in donor cells that have a high level of genetic expression. When used in combination with polymerase chain reaction technology, even rare expression products can be clones. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay, et al., Nucleic Acid Research 11:2325, 1983).

With respect to nucleotide sequences that are within the scope of the invention, all nucleotide sequences encoding the polypeptides that are embodiments of the invention as described are included in nucleotide sequences that are within the scope of the invention. This further includes all nucleotide sequences that encode polypeptides according to the invention that incorporate conservative amino acid substitutions as defined above. This further includes nucleotide sequences that encode larger proteins incorporating the zinc finger domains, including fusion proteins, and proteins that incorporate transcription modulators operatively linked to zinc finger domains.

Nucleic acid sequences of the present invention further include nucleic acid sequences that are at least 95% identical to the sequences above, with the proviso that the nucleic acid sequences retain the activity of the sequences before substitutions of bases are made, including any activity of proteins that are encoded by the nucleotide sequences and any activity of the nucleotide sequences that is expressed at the nucleic acid level, such as the binding sites for proteins affecting transcription. Preferably, the nucleic acid sequences are at least 97.5% identical. More preferably, they are at least 99% identical. For these purposes, “identity” is defined according to the Needteman-Wunsch algorithm (S. B. Needleman & C. D. Wunsch, “A General Method Applicable to the Search for Similarities in the Amino Acid Sequence of Two Proteins,” J. Mol. Biol. 48: 443-453 (1970)).

Nucleotide sequences encompassed by the present invention can also be incorporated into a vector, including, but not limited to, an expression vector, and used to transfect or transform suitable host cells, as is well known in the art. The vectors incorporating the nucleotide sequences that are encompassed by the present invention are also within the scope of the invention. Host cells that are transformed or transfected with the vector or with polynucleotides or nucleotide sequences of the present invention are also within the scope of the invention. The host cells can be prokaryotic or eukaryotic; if eukaryotic, the host cells can be mammalian cells, insect cells, or yeast cells. If prokaryotic, the host cells are typically bacterial cells.

Transformation of a host cell with recombinant DNA may be carried out by conventional techniques as are well known to those skilled in the art. Where the host is prokaryotic, such as Escherichia coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used.

A variety of host-expression vector systems may be utilized to express the zinc finger derived-nucleotide binding coding sequence. These include but are not limited to microorganisms such as bacteria transformed with recombinant bacteriophage DNA, plasmid DNA or cosmid DNA expression vectors containing a zinc finger derived-nucleotide binding polypeptide coding sequence; yeast transformed with recombinant yeast expression vectors containing the zinc finger-nucleotide binding coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a zinc finger derived-DNA binding coding sequence; insect cell systems infected with recombinant virus expression vectors (e.g., baculovirus) containing a zinc finger-nucleotide binding coding sequence; or animal cell systems infected with recombinant virus expression vectors (e.g., retroviruses, adenovirus, vaccinia virus) containing a zinc finger derived-nucleotide binding coding sequence, or transformed animal cell systems engineered for stable expression. In such cases where glycosylation may be important, expression systems that provide for translational and post-translational modifications may be used; e.g., mammalian, insect, yeast or plant expression systems.

Depending on the host/vector system utilized, any of a number of suitable transcription and translation elements, including constitutive and inducible promoters, transcription enhancer elements, transcription terminators, etc. may be used in the expression vector (see e.g., Bitter, et al., Methods in Enzymology, 153:516-544, 1987). For example, when cloning in bacterial systems, inducible promoters such as pL of bacteriophage λ, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the inserted zinc finger-nucleotide binding polypeptide coding sequence.

In bacterial systems a number of expression vectors may be advantageously selected depending upon the use intended for the zinc finger derived nucleotide-binding polypeptide expressed. For example, when large quantities are to be produced, vectors which direct the expression of high levels of fusion protein products that are readily purified may be desirable. Those which are engineered to contain a cleavage site to aid in recovering the protein are preferred. Such vectors include but are not limited to the Escherichia coli expression vector pUR278 (Ruther, et al., EMBO J., 2:1791, 1983), in which the zinc finger-nucleotide binding protein coding sequence may be ligated into the vector in frame with the lac Z coding region so that a hybrid zinc finger-lac Z protein is produced; pIN vectors (Inouye & Inouye, Nucleic Acids Res. 13:3101-3109, 1985; Van Heeke & Schuster, J. Biol. Chem. 264:5503-5509, 1989); and the like.

In yeast, a number of vectors containing constitutive or inducible promoters may be used. For a review see, Current Protocols in Molecular Biology, Vol. 2, 1988, Ed. Ausubel, et al., Greene Publish. Assoc. & Wiley lnterscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (Cloning in: Yeast, Ch. 3, R. Rothstein In: DNA Cloning Vol. II, A Practical Approach, Ed. DM Glover, 1986, IRL Press, Wash., D.C.). Alternatively, vectors may be used which promote integration of foreign DNA sequences into the yeast chromosome.

In cases where plant expression vectors are used, the expression of a zinc finger-nucleotide binding polypeptide coding sequence may be driven by any of a number of promoters. For example, viral promoters such as the 35S RNA and 19S RNA promoters of CaMV (Brisson, et al., Nature, 310:511-514, 1984), or the coat protein promoter to TMV (Takamatsu, et al., EMBO J., 6:307-311, 1987) may be used; alternatively, plant promoters such as the small subunit of RUBISCO (Coruzzi, et al., EMBO J. 3:1671-1680, 1984; Broglie, et al., Science 224:838-843, 1984); or heat shock promoters, e.g., soybean hspl7.5-E or hspl 7.3-B (Gurley, et al., Mol. Cell. Biol., 6:559-565, 1986) may be used. These constructs can be introduced into plant cells using Ti plasmids, Ri plasmids, plant virus vectors, direct DNA transformation, microinjection, electroporation, etc. For reviews of such techniques see, for example, Weissbach & Weissbach, Methods for Plant Molecular Biology, Academic Press, NY, Section VIII, pp. 421-463, 1988; and Grierson & Corey, Plant Molecular Biology, 2d Ed., Blackie, London, Ch. 7-9, 1988.

An alternative expression system that can be used to express a protein of the invention is an Insect system. In one such system, Autographa californica nuclear polyhedrosis virus (AcNPV) is used as a vector to express foreign genes. The virus grows in Spodoptera frugiperda cells. The zinc finger-nucleotide binding polypeptide coding sequence may be cloned into non-essential regions (in Spodoptera frugiperda, for example, the polyhedrin gene) of the virus and placed under control of an AcNPV promoter (for example the polyhedrin promoter). Successful insertion of the zinc finger-nucleotide binding polypeptide coding sequence will result in inactivation of the polyhedrin gene and production of non-occluded recombinant virus (i.e., virus lacking the proteinaceous coat coded for by the polyhedrin gene). These recombinant viruses are then used to infect cells in which the inserted gene is expressed. (E.g., see Smith, et al., J. Biol. 46:584, 1983; Smith, U.S. Pat. No. 4,215,051).

Eukaryotic systems, and preferably mammalian expression systems, allow for proper post-translational modifications of expressed mammalian proteins to occur. Therefore, eukaryotic cells, such as mammalian cells that possess the cellular machinery for proper processing of the primary transcript, glycosylation, phosphorylation, and, advantageously secretion of the gene product, are the preferred host cells for the expression of a zinc finger derived-nucleotide binding polypeptide. Such host cell lines may include but are not limited to CHO, VERO, BHK, HeLa, COS, MDCK, 293, and WI38.

Mammalian cell systems that utilize recombinant viruses or viral elements to direct expression may be engineered. For example, when using adenovirus expression vectors, the coding sequence of a zinc finger derived polypeptide may be ligated to an adenovirus transcription/translation control complex, e.g., the late promoter and tripartite leader sequence. This chimeric gene may then be inserted into the adenovirus genome by in vitro or in vivo recombination. Insertion in a non-essential region of the viral genome (e.g., region E1 or E3) will result in a recombinant virus that is viable and capable of expressing the zinc finger polypeptide in infected hosts (e.g., see Logan & Shenk, Proc. Natl. Acad. Sci. USA 81:3655-3659, 1984). Alternatively, the vaccinia virus 7.5K promoter may be used. (e.g., see, Mackett et al., Proc. Natl. Acad. Sci. USA, 79:7415-7419, 1982; Mackett, et al., J. Virol. 49:857-864, 1984; Panicali, et al., Proc. Natl. Acad. Sci. USA, 79:4927-4931, 1982). Of particular interest are vectors based on bovine papilloma virus which have the ability to replicate as extrachromosomal elements (Sarver, et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression of the zinc finger-nucleotide binding protein gene in host cells (Cone & Mulligan, Proc. Natl. Acad. Sci. USA 81:6349-6353, 1984). High level expression may also be achieved using inducible promoters, including, but hot limited to, the metallothionein IIA promoter and heat shock promoters.

For long-term, high-yield production of recombinant proteins, stable expression is preferred. Rather than using expression vectors which contain viral origins of replication, host cells can be transformed with the a cDNA controlled by appropriate expression control elements (e.g., promoter, enhancer, sequences, transcription terminators, polyadenylation sites, etc.), and a selectable marker, The selectable marker in the recombinant plasmid confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used, including but not limited to the herpes simplex virus thymidine kinase (Wigler, et al., Cell 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy, et al., Cell, 22:817, 1980) genes, which can be employed in tk⁻, hgprt⁻ or aprt⁻ cells respectively. Also, antimetabolite resistance-conferring genes can be used as the basis of selection; for example, the genes for dhfr, which confers resistance to methotrexate (Wigler, et al., Natl. Acad. Sci. USA, 77:3567, 1980; O'Hare, et al., Proc. Nat[. Acad. Sci. USA, 78:1527, 1981); gpt, which confers resistance to mycophenolic acid (Mulligan & Berg, Proc. Natl. Acad. Sci. USA, 78:2072, 1981; neo, which confers resistance to the aminoglycoside G418 (Colberre-Garapin, et al., J. Mol. Biol., 150:1, 1981); and hygro, which confers resistance to hygromycin (Santerre, et al., Gene, 30:147, 1984). Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:804, 1988); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory ed., 1987).

Isolation and purification of microbially expressed protein, or fragments thereof provided by the invention, may be carried out by conventional means including preparative chromatography and immunological separations involving monoclonal or polyclonal antibodies. Antibodies provided in the present invention are immunoreactive with the zinc finger-nucleotide binding protein of the invention. Antibody which consists essentially of pooled monoclonal antibodies with different epitopic specificities, as well as distinct monoclonal antibody preparations are provided. Monoclonal antibodies are made from antigen containing fragments of the protein by methods well known in the art (Kohler, et al., Nature, 256:495, 1975; Current Protocols in Molecular Biology, Ausubel, et al., ed., 1989).

VI. Pharmaceutical Compositions

In another aspect, the present invention provides a pharmaceutical composition comprising:

(1) a therapeutically effective amount of a polypeptide, polypeptide composition, or isolated heptapeptide according to the present invention as described above; and

(2) a pharmaceutically acceptable carrier.
Alternatively, the present invention also provides:

(1) a therapeutically effective amount of a nucleotide sequence that encodes a polypeptide, polypeptide composition, or isolated heptapeptide according to the present invention as described above; and

(2) a pharmaceutically acceptable carrier.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified. The active ingredient can be mixed with excipients that are pharmaceutically acceptable and compatible with the active ingredient and in amounts suitable for use in the therapeutic methods described herein Suitable excipients are, for example, water, saline, dextrose, glycerol, ethanol or the like and combinations thereof. In addition, if desired, the composition can contain minor amounts of auxiliary substances such as wetting or emulsifying agents, as well as pH buffering agents and the like which enhance the effectiveness of the active ingredient. Still other ingredients that are conventional in the pharmaceutical art, such as chelating agents, preservatives, antibacterial agents, antioxidants, coloring agents, flavoring agents, and others, can be employed depending on the characteristics of the composition and the intended route of administration for the composition.

The pharmaceutical composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylaminoethanol, histidine, procaine and the like. Physiologically acceptable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes. Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

VII. Uses

In one embodiment, a method of the invention includes a process for modulating (inhibiting or suppressing) expression of a nucleotide sequence that contains an AGC target sequence. The method includes the step of contacting the nucleotide with an effective amount of a zinc finger-nucleotide binding polypeptide of this invention that binds to the motif. In the case where the nucleotide sequence is a promoter, the method includes inhibiting the transcriptional transactivation of a promoter containing a zinc finger-DNA binding motif. The term “inhibiting” refers to the suppression of the level of activation of transcription of a structural gene operably linked to a promoter, containing a zinc finger-nucleotide binding motif, for example. In addition, the zinc finger-nucleotide binding polypeptide can bind a target within a structural gene or within an RNA sequence.

The term “effective amount” includes that amount which results in the deactivation of a previously activated promoter or that amount which results in the inactivation of a promoter containing a target ndcleotide, or that amount which blocks transcription of a structural gene or translation of RNA. The amount of zinc finger derived-nucleotide binding polypeptide required is that amount necessary to either displace a native zinc finger-nucleotide binding protein in an existing protein/promoter complex, or that amount necessary to compete with the native zinc finger-nucleotide binding protein to form a complex with the promoter itself. Similarly, the amount required to block a structural gene or RNA is that amount which binds to and blocks RNA polymerase from reading through on the gene or that amount which inhibits translation, respectively. Preferably, the method is performed intracellularly. By functionally inactivating a promoter or structural gene, transcription or translation is suppressed. Delivery of an effective amount of the inhibitory protein for binding to or “contacting” the cellular nucleotide sequence containing the target sequence can be accomplished by one of the mechanisms described herein, such as by retroviral vectors or liposomes, or other methods well known in the art. The term “modulating” refers to the suppression, enhancement or induction of a function. For example, the zinc finger-nucleotide binding polypeptide of the invention can modulate a promoter sequence by binding to a target sequence within the promoter, thereby enhancing or suppressing transcription of a gene operatively linked to the promoter nucleotide sequence. Alternatively, modulation may include inhibition of transcription of a gene where the zinc finger-nucleotide binding polypeptide binds to the structural gene and blocks DNA dependent RNA polymerase from reading through the gene, thus inhibiting transcription of the gene. The structural gene may be a normal cellular gene or an oncogene, for example. Alternatively, modulation may include inhibition of translation of a transcript.

The promoter region of a gene includes the regulatory elements that typically lie 5′ to a structural gene; multiple regulatory elements can be present, separated by intervening nucleotide sequences. If a gene is to be activated, proteins known as transcription factors attach to the promoter region of the gene. This assembly resembles an “on switch” by enabling an enzyme to transcribe a second genetic segment from DNA to RNA. In most cases the resulting RNA molecule serves as a template for synthesis of a specific protein; sometimes RNA itself is the final product.

The promoter region may be a normal cellular promoter or, for example, an onco-promoter. An onco-promoter is generally a virus-derived promoter. For example, the long terminal repeat (LTR) of retroviruses is a promoter region that may be a target for a zinc finger binding polypeptide variant of the invention. Promoters from members of the Lentivirus group, which include such pathogens as human T-cell lymphotrophic virus (HTLV) 1 and 2, or human immunodeficiency virus (HIV) 1 or 2, are examples of viral promoter regions which may be targeted for transcriptional modulation by a zinc finger binding polypeptide of the invention

A target AGC nucleotide sequence can be located in a transcribed region of a gene or in an expressed sequence tag. As described above, the target AGC sequence can also be located adjacent to the transcription termination site of a gene. A gene containing a target sequence can be a plant gene, an animal gene or a viral gene. The gene can be a eukaryotic gene or prokaryotic gene such as a bacterial gene. The animal gene can be a mammalian gene including a human gene. In a preferred embodiment, a method of modulating nucleotide expression is accomplished by transforming a cell that contains a target nucleotide sequence with a polynucleotide that encodes a polypeptide or composition of this invention. Preferably, the encoding polynucleotide is contained in an expression vector suitable for use in a target cell. Suitable expression vectors are well known in the art.

The AGC target can exist in any combination with other target triplet sequences. That is, a particular AGC target can exist as part of an extended AGC sequence (e.g., [AGC]₂₋₁₂) or as part of any other extended sequence such as (GNN)₁₋₁₂, (ANN)₁₋₁₂, (CNN)₁-₁₂, (TNN)₁₋₁₂or (NNN)₁₋₁₂.

The Examples that follow illustrate preferred embodiments of the present invention and are not limiting of the specification and claims in any way.

### EXAMPLE 1

Construction of Zinc Finger Library and Selection via Phage Display

Introduction

Cys₂-His₂ zinc finger proteins are one of the most common DNA-binding motifs found in eukaryotic transcription factors. These zinc fingers are compact domains containing a single amphipathic α-helix stabilized by two β-strands and zinc ligation. Amino acids on the surface of the α-helix contact bases in the major groove of DNA. Zinc finger proteins typically contain multiple fingers that make tandem contacts along the DNA. The mode of DNA recognition is principally a one-to-one interaction between amino acids from the recognition helix and DNA bases. One finger usually recognizes 3 base pairs (bp). As these fingers function as independent modules, fingers with different triplet specificities can be combined to give specific recognition of longer DNA sequences. This simple, modular structure of zinc finger domains and the wide variety of DNA sequences they can recognize make them an attractive framework for the design of novel DNA-binding proteins.

The ability to rapidly prepare proteins with predefined specificities for DNA sequences could enable a wide range of technologies that might be used for example to direct the expression of genes or to physically modify genes and genomes. In order to develop a universal system for gene regulation, much effort has been applied to the development of artificial transcription factors based on polydactyl zinc finger proteins (Blancafort, P., Segal, D. J., and Barbas, C. F., 3rd. (2004) Mol Pharmacol 66(6), 1361-1371; Beerli, R. R., and Barbas, C. F., 3rd. (2002) Nat Biotechnol 20(2), 135-141; Jantz, D., and Berg, J. M. (2004) Chem Rev. 104(2), 789-799). Such a system might have considerable impact on biology and biotechnology and offer a new approach for treatment of diseases based on directed gene regulation. It has now been shown that gene expression can be specifically altered using artificial transcription factors based on polydactyl zinc finger proteins that bind to 18 base pair (bp) target sites (Blancafort, P., Segal, D. J., and Barbas, C. F., 3rd. (2004) Mol Pharmacol 66(6), 1361-1371; Beerli, R. R., and Barbas, C. F., 3rd. (2002) Nat Biotechnol 20(2), 135-141). Targeting of sites as small as 9 bp can also provide some degree of regulatory specificity presumably through the aid of chromatin occlusion (Zhang, L., Spratt, S. K., Liu, Q., Johnstone, B., Qi, H., Raschke, E. E., Jamieson, A. C., Rebar, E. J., Wolffe, A. P., and Case, C. C. (2000) J Biol Chem 275(43), 33850-33860; Liu, P. Q., Rebar, E. J., Zhang, L., Liu, Q., Jamieson, A. C., Liang, Y., Qi, H., Li, P. X., Chen, B., Mendel, M. C., Zhong, X., Lee, Y. L., Eisenberg, S. P., Spratt, S. K., Case, C. C., and Wolffe, A. P. (2001) J Biol Chem 276(14), 11323-11334; Blancafort, P., Magnenat, L., and Barbas, C. F., 3rd. (2003) Nat Biotechnol 21(3), 269-274). In addition to transcriptional regulation, novel zinc finger DNA-binding specificities are showing tremendous promise in directing homologous recombination through their fusion with the Fok I nuclease domain (Urnov F D, M. J., Lee Y L, Beausejour C M, Rock J M, Augustus S, Jamieson A C, Porteus M H, Gregory P D, Holmes M C. (2005) Nature 435(7042), 646-651; Bibikova, M., Beumer, K., Trautman, J. K., and Carroll, D. (2003) Science 300(5620), 764).

Zinc finger domains of the type Cys₂-His₂ are a unique and promising class of proteins for the recognition of extended DNA sequences due to their modular nature. Each domain consists of approximately 30 amino acids folded into a ββα structure stabilized by hydrophobic interactions and chelation of a zinc ion by the conserved Cys₂-His₂ residues (Miller, J., McLachlan, A. D., and Klug, A. (1985) EMBO J. 4(6), 1609-1614; Lee, M. S., Gippert, G. P., Soman, K. V., Case, D. A., and Wright, P. E. (1989) Science (Washington, D. C., 1883-) 245(4918), 635-637). To date, the best-characterized protein of this family of zinc finger proteins is the mouse transcription factor Zif268. Each of the three zinc finger domains of Zif268 binds to a 3 bp subsite by insertion of the α-recognition helix into the major groove of the DNA double helix (Pavletich, N. P., and Pabo, C. O. (1991) Science (Washington, D. C., 1883-) 252(5007), 809-817; Elrod-Erickson, M., Rould, M. A., Nekludova, L., and Pabo, C. O. (1996) Structure 4, 1171-1180). To facilitate the rapid construction of DNA-binding proteins and to study protein-DNA interactions, domains have previously been created that bind to the 5′-GNN-3′ and 5′-ANN-3′ family of DNA sequences (Segal, D. J., Dreier, B., Beerli, R. R., and Barbas, C. F., 3rd. (1999) Proc NatlAcad Sci U S A 96(6), 2758-2763; Dreier, B., Segal, D. J., and Barbas, C. F., 3rd. (2000) J Mol Biol 303(4), 489-502; Dreier, B., Beerli, R. R., Segal, D. J., Flippin, J. D., and Barbas, C. F., 3rd. (2001) J Biol Chem 276(31), 29466-29478). It was demonstrated that these domains function as modular recognition units that can be assembled into polydactyl zinc finger proteins that specifically recognize from 9 to 18 bp target sites. Significantly, an 18 bp site is long enough to potentially be unique within the human, or any other genome and transcriptional specificity of such proteins has been demonstrated in transgenic plants and human cells using array analysis (Guan, X., Stege, J., Kim, M., Dahmani, Z., Fan, N., Heifetz, P., Barbas, C. F., 3rd, and Briggs, S. P. (2002) Proc Natl Acad Sci U S A 99(20), 13296-13301; Tan, S., Guschin, D., Davalos, A., Lee, Y. L., Snowden, A. W., Jouvenot, Y., Zhang, H. S., Howes, K., McNamara, A. R., Lai, A., Ullman, C., Reynolds, L., Moore, M., Isalan, M., Berg, L. P., Campos, B., Qi, H., Spratt, S. K., Case, C. C., Pabo, C. O., Campisi, J., and Gregory, P. D. (2003) Proc. Nail. Acad. Sci., U S A. 100(21), 11997-12002). In addition to constitutive regulation, fusion of ligand-binding domains from nuclear hormone receptors with specifid binding domains provides inducible gene regulation with this class of transcription factors (Beerli, R. R., Schopfer, U., Dreier; B., and Barbas, C. F., 3rd. (2000) J Biol Chem 275(42), 32617-32627). To provide for ultimate freedom in DNA targeting it is important to identify the 64 DNA-binding domains required to target each possible 3-bp subsite.

Due to the limited structural data on zinc finger/DNA interactions (Pavietich, N. P., and Pabo, C. O. (1993) Science (Washington, D. C., 1883-) 261(51.29), 1701-1707; Kim, C. A., and Bergs J. M. (1996) Nature Structural Biology 3, 940-945; Fairall, L., Schwabe, , J. W. R., Chapman, L., Finch, J. T., and Rhodes, D. (1993) Nature (London) 366(6454), 483-487; Houbaviy, H. B., Usheva, A., Shenk, T., and Burley, S. K. (1996) Proc. Natl. Acad. Sci. U. S. A. 93(24), 13577-13582; Wuttke, D. S., Foster, M. P., Case, D. A., Gottesfeld, J. M., and Wright, P. E. (1997) J. Mol BioL 273(1.), 183-206; Nolte, R. T., Conlin, R. M., Harrison, S. C., and Brown, R. S. (1998) Proc. Natl. Acad. Sci. U. S. A. 95(6), 2938-2943) de novo design of zinc proteins that bind with a high degree of specificity to novel sequences has been of limited success (Havranek J J, D. C., Baker D. (2004) J Mol Biol. 344(1), 59-70). Crystallographic data and mutagenesis studies concerning the mode of interaction of zinc finger domains of the Cys₂-His₂ family has guided us in the construction of phage display libraries for selection of domains that recognize many DNA subsites (Dreier, B., Beerli, R. R., Segal, D. J., Flippin, J. D., and Barbas, C. F., 3rd. (2001) J Biol Chem 276(31), 29466-29478). The analysis of the Zif268/DNA complex suggests that DNA binding is predominantly achieved by the interaction of amino acid residues of the α-helix in positions −1, 3, and 6 with the 3′, middle, and 5′ nucleotides of a 3 bp DNA subsite, respectively (Pavletich, N. P., and Pabo, C. O. (1991) Science (Washington, D. C., 1883-) 252(5007), 809-817; Elrod-Erickson, M., Rould, M. A., Nekludova, L., and Pabo, C. O. (1996) Structure 4, 1171-1180). Positions 1, 2, and 5 of the α-helix make direct or water-mediated contacts with the phosphate backbone of the DNA and are important contributors to the ultimate specificity of the protein. Leucine is typically found in position 4 and packs into the hydrophobic core of the domain. Position 2 of the α-helix interacts with other helix residues and, in addition, can make contact with a nucleotide outside the 3 bp subsite resulting in target site overlap (Segal, D. J., Dreier, B., Beerli, R. R., and Barbas, C. F., 3rd. (1999) Proc Natl Acad Sci U S A 96(6), 2758-2763; Dreier, B., Beerli, R. R., Segal, D. J., Flippin, J. D., and Barbas, C. F., 3rd. (2001) J Biol Chem 276(31), 29466-29478; Wolfe S A, G. H., Ramm El, Pabo C O. (1999) J Mol Biol 285(5), 1917-1934; Isalan, M., Choo, Y., and Klug, A. (1997) Proc. Natl. Acad. Sci. U. S. A. 94(11), 5617-5621; Pabo C.O., Nekludova, L. (2000) J Mol Biot 301(3), 597-624).

The most studied scaffold for building proteins of novel specificity have been the murine transcription factor Zif268 and the structurally related human transcription factor Sp1.

FIG. 1 shows the zinc finger-DNA complex of the murine transcription factor Zif268.

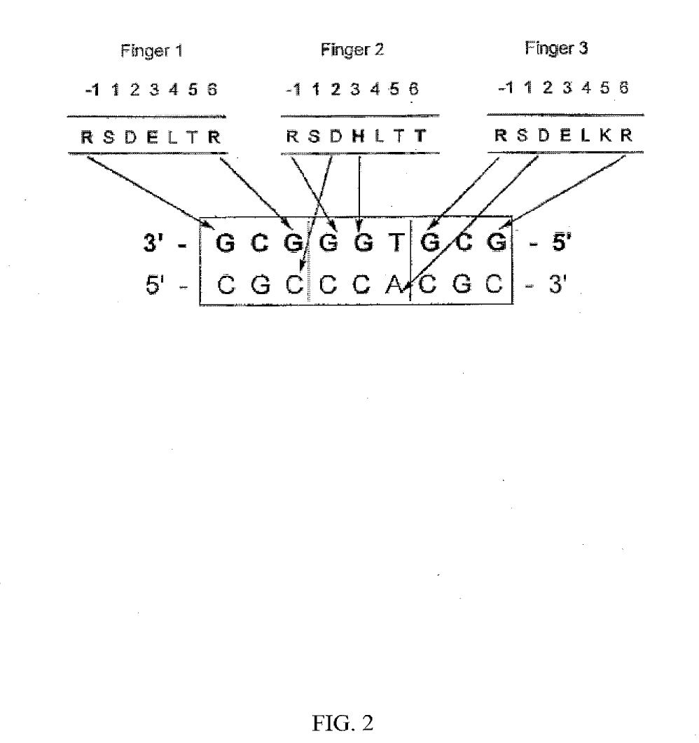

The structure and DNA-binding specificity of both proteins are well-studied (Elrod-Erickson, M., Rould, M. A., Nekludova, L., and Pabo, C. O. (1996) Structure 4, 1171-1180; Narayan, V. A, Kriwacki, R. W., and Caradonna, J. P. (1997), J. Biol Chem. 272, 7801-7809). FIG. 2 shows the protein-DNA interaction of the transcription factor Zif268 in terms of the interaction between specific bases of the DNA and specific amino acids of the three fingers of the transcription factor. Positions −1, 3, and −6 were generally observed to contact the 3′-, middle, and 5-′nucleotides of a base triplet, respectively. Positions −2, 1, and 5 are often involved in direct or water mediated contacts to the phosphate backbone. Position 4 is typically a leucine residue that packs in the hydrophobic core of the domain. Position 2 has been shown to interact with other helix residues and/or bases depending on the helix structure. In the Zif268-DNA complex aspartate at position 2 of finger 2 and in position 2 of finger 3 contacts cytosine or adenine, respectively, on the complementary DNA strand, which is called “target site overlap.” Distinguished from other zinc finger binding proteins Zif268 and Sp1 show only low inter-domain cooperative binding activity, which make them attractive frameworks for investigation of zinc finger structure-activity relationships and for the design of novel zinc finger domains.

However, the structural details of recognition are still complicated to define. The selection of zinc-finger domains which had been characterized in detail to specifically bind to DNA focused so far on the 5′-(GNN)-3′ target family. Some information about amino acid-base interactions in detail from this work is provided in Table 1.

Most of the successful selections have involved sites of this form. For the majority of the remaining 48 triplets, only a few fingers with the desired specificity have been reported. It is not yet known to what extent this represents an intrinsic preference of zinc fingers for binding to 5′-(GNN)-3′ targets or just the limited target sites which have been tested so far. According to the fact that “cross-strand” interactions from position 2 to the neighboring base pair on the adjacent triplet can influence the specificity of binding, the simple model that zinc fingers are essentially independent modules binding three base pairs has to be revised to a model that considers synergy between adjacent fingers. The construction of multi-finger proteins remains challenging not only because of the inter-domain cooperativity but also because effects of the linker region and the β-strands of the zinc finger protein structure have to be considered. The goal of the work reported in this Example is to select zinc finger domains which bind specifically to 5′-(TNN)-3′ DNA sequences. To date, recognition of the 5′-nucleotide by the amino acid in position 6 of the α-helix is not understood, except the interaction of the 5′-guanine with arginine or lysine (Table 1).

Construction of Zinc Finger Library and Selection via Phage Display

Construction of the zinc finger library was based on the earier described C7 protein ([Wu et al., (1995) PNAS 92, 344-348]). Finger 3 recognizing the 5′-GCG-3′ subsite was replaced by a domain binding to a 5′-GAT-3′ subsite [Segal et al., (1999) Proc Natl Acad Sci USA 96(6), 2758-2763] via a overlap PCR strategy using a primer coding for finger 3 (5′-GAGGAAGTTTGCCACCAGTGGCAACCTGGTGAGGCATACCAAAATC-3′) (SEQ ID NO: 111 ) and a pMa1-specific primer (5′-GTAAAACGACGGCCAGTGCCAAGC-3′) (SEQ ID NO: 112). Randomization of the zinc finger library by PCR overlap extension was essentially as described [Wu et al., (1995) PNAS92, 344-348; Segal et al., (1999) Proc Natl Acad Sci USA 96(6), 2758-2763] The library was ligated into the phagemid vector pComb3H [Rader et al., (1997) Curr. Opin. Biotechnol. 8(4), 503-508]. Growth and precipitation of phage were performed as previously described [Barbas et al., (1991) Methods: Companion Methods Enzymol. 2(2), 119-124; Barbas et al., (1991) Proc. Natl. Acad. Sci. USA 88, 7978-7982; Segal et al., (1999) Proc Natl Acad Sci USA 96(6), 2758-2763]. Binding reactions were performed in a volume of 500 μl zinc buffer A (ZBA: 10 mM Tris, pH 7.5/90 mM KCl/1 mM MgCl₂/90 μM ZnCl.sub.2)/0.2% BSA/5 mM DTT/1% Blotto (Biorad)/20 μg double-stranded, sheared herring sperm DNA containing 100 μl precipitated phage (10¹³ colony-forming units). Phage were allowed to bind to non-biotinylated competitor oligonucleotides for 1 hr at 4° C. before the biotinylated target oligonucleotide was added. Binding continued overnight at 4° C. After incubation with 50 μl streptavidin coated magnetic beads (Dynal; blocked with 5% Blotto in ZBA) for 1 hr, beads were washed ten times with 500 μl ZBA/2% Tween 20/5 mM DTT, and once with buffer containing no Tween. Elution of bound phage was performed by incubation in 25 μl trypsin (10 mg/ml) in TBS (Tris-buffered saline) for 30 min at room temperature. Hairpin competitor oligonucleotides had the sequence 5′-GGCCGCN′N′N′ATCGAGTTTTCTCGATNNNGCGGCC-3′ (SEQ ID NO: 113) (target oligonucleotides were biotinylated), where NNN represents the finger-2 subsite oligonucleotides, N′N′N′ its complementary bases. Target oligonucleotides were usually added at 72 nM in the first three rounds of selection, then decreased to 36 nM and 18 nM in the sixth and last round. As competitor a 5′-TGG-3′ finger-2 subsite oligonucleotide was used to compete with the parental clone. An equimolar mixture of 15 finger-2 5′-ANN-3′ subsites, except for the target site, respectively, and competitor mixtures of each finger-2 subsites of the type 5′-CNN-3′, 5′-GNN-3′, and 5′-TNN-3′ were added in increasing amounts with each successive round of selection. Usually no specific 5′-ANN-3′ competitor mix was added in the first round.

Multitarget Specificity Assay and Gel Mobility Shift Analysis

The zinc finger-coding sequence was subcloned from pComb3H into a modified bacterial expression vector pMal-c2 (New England Biolabs). After transformation into XL1-Blue (Stratagene) the zinc finger-maltose-binding protein (MBP) fusions were expressed after addition of 1 nM isopropyl β-D-thiogalactoside (IPTG). Freeze/thaw extracts of these bacterial cultures were applied in 1:2 dilutions to 96-well plates coated with streptavidin (Pierce), and were tested for DNA-binding specificity against each of the sixteen 5′-GAT ANN GCG-3′ (SEQ ID NO: 114) target sites, respectively. ELISA (enzyme-linked immunosorbent assay) was performed essentially as described [Segal et al., (1999) Proc Natl Acad Sci USA 96(6), 2758-2763; Dreier et al., (2000) J. Mol. Biol 303, 489-502]. After incubation with a mouse anti-MBP (maltose-binding protein) antibody (Sigma, 1:1000), a goat anti-mouse antibody coupled with alkaline phosphatase (Sigma, 1:1000) was applied. Detection followed by addition of alkaline phosphatase substrate (Sigma), and the OD405 was determined with SOFTMAX2.35 (Molecular Devices).

### EXAMPLE 2

Site-directed Mutagenesis of Finger 2

Finger-2 mutants were constructed by PCR as described [Segal et al., (1999) Proc Natl Acad Sci USA 96(6), 2758-2763; Dreier et al., (2000) J. Mol. Biol. 303, 489-502]. As PCR template the library clone containing 5′-TGG-3′ finger 2 and 5′-GAT-3′ finger 3 was used. PCR products containing a mutagenized finger 2 and 5′-GAT-3′ finger 3 were subcloned via Nsil and Spel restriction sites in frame with finger 1 of C7 into a modified pMal-c2 vector (New England Biolabs).

Construction of Polydactyl Zinc Finger Proteins

Three-finger proteins were constructed by finger-2 stitchery using the SP1 C framework as described [Beerli et al., (1998) Proc Natl Acad Sci USA 95(25), 14628-14633]. The proteins generated in this work contained helices recognizing 5′-GNN-3′ DNA sequences [Segal et al., (1999) Proc Natl Acad Sci USA 96(6), 2758-2763], as well as 5′-ANN-3′ and 5′-TAG-3′ helices described here. Six finger proteins were assembled via compatible Xmal and BsrFl restriction sites. Analysis of DNA-binding properties were performed from IPTG-induced freeze/thaw bacterial extracts. For the analysis of capability of these proteins to regulate gene expression they were fused to the activation domain VP64 or repression domain KFRAB of Kox-1 as described earlier ([Beerli et al., (1998) Proc Natl Acad Sci USA 95(25), 14628-14633; Beerli et al., (2000) Proc Natl Acad Sci USA 97(4), 1495-1500; Beerli et al., (2000) J. Biol. Chem. 275(42), 32617-32627]; VP64: tetrameric repeat of herpes simplex virus' VPI6 minimal activation domain) and subcloned into pcDNA3 or the retroviral pMX-IRES-GFP vector ([Liu et al., (1997) Proc. Natl. Acad. Sci. USA 94, 10669-10674]; IRES, internal ribosome-entry site; GFP, green fluorescent protein).

### EXAMPLE 3

Design of New Randomized Zinc Finger Libraries with Changed Linker Regions

Introduction

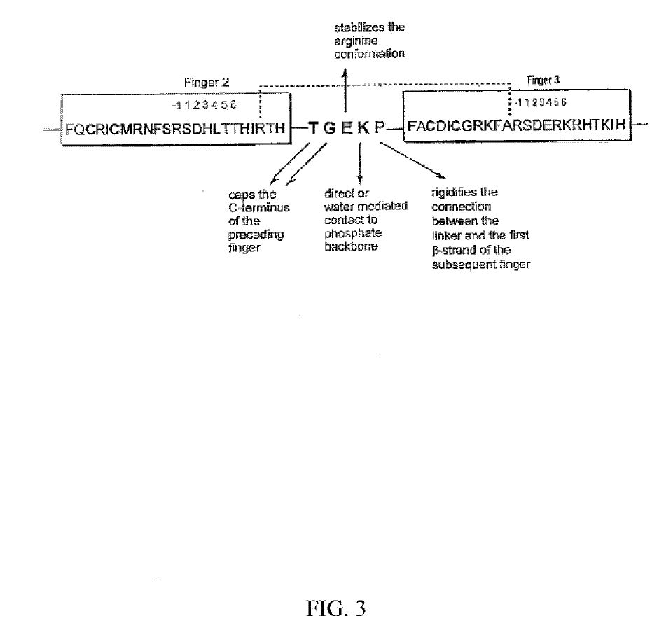

The linker region that connects neighboring zinc fingers is an important structural element that helps control the spacing of the fingers along the DNA site. The most common linker arrangement has five residues between the final histidine of one finger and the first conserved aromatic amino acid of the next finger. Roughly half of the linkers of zinc fingers found in the Transcription Factor Database conform to the consensus sequence TGEKP (SEQ ID NO: 100). The structural role of each of the linker residues has already been examined (FIG. 3). The docking of adjacent fingers is further stabilized by contact between the side chain of position 9 of the preceding finger's helix and the backbone carbonyl or side chain at position −2 of the subsequent finger. This contact can be correlated with the TGEKP (SEQ ID NO: 100) linker. Whenever it occurs between zinc fingers there are almost always three residues between the two histidines of the preceding finger, and in 80% of these proteins there is a basic amino acid (arginine or lysine) at position 9. When arginine occurs in this position, it makes an interfinger contact with the backbone carbonyl at position −2. In some structures, the conformation of this arginine has been found to be stabilized by an interaction with glutamate from the linker.

Mutagenesis studies have demonstrated that the linker sequence is important for high-affinity DNA binding. Some point mutations result in 10-100 fold decrease of DNA binding affinity and can lead to a loss of function in vivo. NMR studies indicate that the TGEKP (SEQ ID NO: 100) linker is flexible in the free protein, but becomes more rigid upon binding to DNA.

Cys₂-His₂ zinc finger proteins often bind their target sites with high affinity and specificity. Several groups have noted that as the number of TGEKP (SEQ ID NO: 100)-linked fingers increases from one to two to three, there is an accompanying increase in DNA-binding affinity. Proteins containing three fingers, such as Zif268 and SP1, bind their preferred sequences with dissociation constants typically between 10⁻⁸ M and 10⁻¹¹ M. Unexpectedly the attachment of additional fingers using the TGEKP (SEQ ID NO: 100) linker leads only to modest additional increase of binding affinity to DNA. The reasons for that are not entirely clear and further studies are needed to understand the basis of this effect. The structural and energetic problems arising from the presence of four or more fingers in a multifinger protein may arise from the distortion of the DNA molecule that is caused by zinc fingers upon binding to DNA. Zinc fingers connected by TGEKP (SEQ ID NO: 100) linkers adopt a helical arrangement when bound to DNA that does not perfectly match the helical pitch of the DNA, so that as more fingers are attached, more steric hindrance accumulates. The negative energetic consequences of steric hindrance therefore weaken the binding affinity from what it would be in the absence of steric hindrance. Studies of supercoiling levels have shows that zinc finger binding unwinds the DNA by approximately 18° per finger. In the resulting complex, DNA assumes a variant B-form conformation with about 11 base pairs per turn and an enlarged major groove.

There were two approaches which have been used so far to generate polydactyl zinc finger proteins that bind specifically and with high affinity to their DNA targets. One of them is the insertion of a longer, flexible linker between two sets of canonically linked fingers, which would be a covalent arrangement. A six-finger construct consisting of two three-finger proteins derived from Zif268 and NRE connected by a longer, flexible linker showed a femtomolar dissociation constant. Another possibility is the attachment of a dimerization domain onto a canonical set of zinc fingers. The dimerization domain induces the assembly of zinc fingers to a larger complex and thereby the recognition of a longer DNA target site. This approach is fully modular as the stability of the dimer can be influenced which allows, e.g., a tuning of the on and off states.

Design Concept

Design strategies for polydactyl zinc finger proteins which all used canonical linkers to connect the additional fingers, gave relatively modest increased in DNA-binding affinity. Structural and biochemical analysis show that DNA is often slightly unwound when bound to zinc finger peptides. Modeling studies showed that the canonical linker is a bit too short to allow favorable docking, e.g., of Zif268 on ideal B-DNA. The reason for this is that the helical periodicity of the zinc fingers does not quite match the helical periodicity of B-DNA and the strain of unwinding becomes a more serious problem when more fingers are used; this has the effect of reducing the binding affinity because binding becomes energetically relatively less favorable.

It was decided to study the influence of the structure of the linker region on the DNA-binding affinity of polydactyl zinc finger proteins using phage display. Therefore, two different polydactyl zinc finger proteins were chosen, B3C2 and Vegf 5′16; both are six-finger proteins with a DNA binding affinity of about 1 nm.

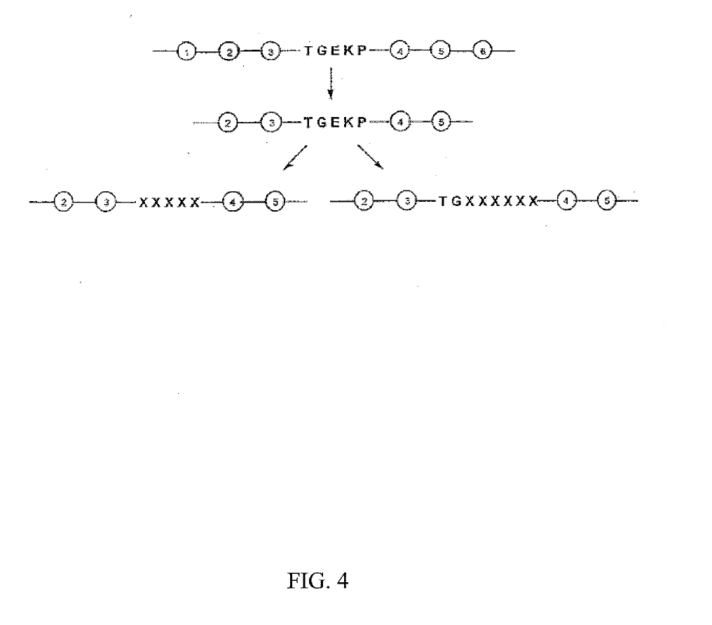

Two different kinds of libraries for each of the peptides were constructed. The first one randomized the five positions of the canonical linker TGEKP (SEQ ID NO: 100) to select variants with changed amino acid sequence that might be less constrained and might be able to bind tighter to DNA. A longer, more flexible linker was also desired. The second set of libraries kept the T and G in the canonical linker TGEKP (SEQ ID NO: 100), randomized the third, fourth, and fifth positions and added three additional amino acids (FIG. 4). Four-finger proteins (containing fingers 2-5) were constructed from the six-finger proteins to make the library construction easier. These four-finger proteins were taken as templates for the PCR to construct the randomized libraries.

### EXAMPLE 4

Gel Mobility Shift Analysis

(Prospective Example)

Gelshift analysis is performed with purified protein (Protein Fusion and Purification System, New England Biolabs) essentially as described In general, fusion proteins are purified to >90% homogeneity using the Protein Fusion and Purification System (New England Biolabs), except that ZBA/5 mM DTT is used as the column buffer. Protein purity and concentration are determined from. Coomassie blue-stained 15% SDS-PAGE gels by comparison to BSA standards. Target oligonucleotides are labeled at their 5′ or 3′ ends with [³²P] and gel purified. Eleven 3-fold serial dilutions of protein are incubated in 20 μl binding reactions (1×Binding Buffer/10% glycerol/≈1 pM target oligonucleotide) for three hours at room temperature, then resolved on a 5% polyacrlyamide gel in 0.5×TBE buffer. Quantitation of dried gels is performed using a Phosphorimager and ImageQuant software (Molecular Dynamics), and the K_{D} is determined by Scatchard analysis.

### EXAMPLE 5

General Methods

(Prospective Example)

Transfection and Luciferase Assays

HeLa cells are used at a confluency of 40-60%. Cells are transfected with 160 ng reporter plasmid (pGL3-promoter constructs) and 40 ng of effector plasmid (zinc finger-effector domain fusions in pcDNA3) in 24 well plates. Cell extracts are prepared 48 hrs after transfection and measured with luciferase assay reagent (Promega) in a MicroLumat LB96P luminometer (EG & Berthold, Gaithersburg, Md.).

Retroviral Gene Targeting and Flow Cytometric Analysis

These assays are performed as described [Beerli et al., (2000) Proc Natl Acad Sci U S A 97(4), 1495-1500; Beerli et al., (2000) J. Biol. Chem. 275(42), 32617-32627]. As primary antibody an ErbB-1 -specific mAb EGFR (Santa Cruz), ErbB-2-specific mAb FSP77 (gift from Nancy E. Hynes; Harwerth et al., 1992) and an ErbB-3-specific mAb SGP1 (Oncogene Research Products) are used. Fluorescently labeled donkey F(ab′)₂ anti-mouse IgG is used as secondary antibody (Jackson lmmuno-Research).

### EXAMPLE 6

Construction of Zinc Finger-Effector Domain Fusion Proteins

(Prospective Example)

For the construction of zinc finger-effector domain fusion proteins, DNAs encoding amino acids 473 to 530 of the ets repressor factor (ERF) repressor domain (ERD) (Sgouras, D. N., Athanasiou, M. A., Beal, G. J., Jr., Fisher, R. J., Blair, D. G. & Mavrothalassitis, G. J. (1995) EMBO J. 14, 4781-4793), amino acids 1 to 97 of the KRAB domain of KOX1 (Margolin, J. F., Friedman, J. R., Meyer, W., K.-H., Vissing, H., Thiesen, H.-J. & Rauscher III, F. J. (1994) Proc. Natl. Acad. Sci. USA 91, 4509-4513), or amino acids 1 to 36 of the Mad mSIN3 interaction domain (SID) (Ayer, D. E., Laherty, C. D., Lawrence, Q. A., Armstrong, A. P. & Eisenman, R. N. (1996) Mol. Cell. Biol. 16, 5772-5781) are assembled from overlapping oligonucleotides using Taq DNA polymerase. The coding region for amino acids 413 to 489 of the VP16 transcriptional activation domain (Sadowski, I., Ma, J., Triezenberg, S. & Ptashne, M. (1988) Nature 335, 563-564) is PCR amplified from pcDNA3/C₇-C₇-VP16 (10). The VP64 DNA, encoding a tetrameric repeat of VP16's minimal activation domain, comprising amino acids 437 to 447 (Seipel, K., Georgiev, O. & Schaffner, W. (1992) EMBO J. 11, 4961-4968), is generated from two pairs of complementary oligonucleotides. The resulting fragments are fused to zinc finger coding regions by standard cloning procedures, such that each resulting construct contained an internal SV40 nuclear localization signal, as well as a C-terminal HA decapeptide tag. Fusion constructs are cloned in the eukaryotic expression vector pcDNA3 (Invitrogen).

### EXAMPLE 7

Construction of Luciferase Reporter Plasmids

(Prospective Example)

An erbB-2 promoter fragment comprising nucleotides −758 to −1, relative to the ATG initiation codon, is PCR amplified from human bone marrow genornic DNA with the TaqExpand DNA polymerase mix (Boehringer Mannheim) and cloned into pGL3basic (Promega), upstream of the firefly luciferase gene. A human erbB-2 promoter fragment encompassing nucleotides −1571 to −24, is excised from pSVOALD5′/erbB-2(N—N) (Hudson, L. G., Ertl, A. P. & Gill, G. N. (1990) J. Biol. Chem. 265, 4389-4393) by Hind3 digestion and subcloned into pGL3basic, upstream of the firefly luciferase gene.

### EXAMPLE 8

Luciferase Assays

(Prospective Example)

For all transfections, HeLa cells are used at a confluency of 40-60%. Typically, cells are transfected with 400 ng reporter plasmid (pGL3-promoter constructs or, as negative control, pGL3basic), 50 ng effector plasmid (zinc finger constructs in pcDNA3 or, as negative control, empty pcDNA3), and 200 ng internal standard plasmid (phrAct-bGal) in a well of a 6 well dish using the lipofectamine reagent (Gibco BRL). Cell extracts are prepared approximately 48 hours after transfection. Luciferase activity is measured with luciferase assay reagent (Promega), PGal activity with Galacto-Light (Tropix), in a MicroLumat LB 96P luminometer (EG&G Berthold). Luciferase activity is normalized on βGal activity.

### EXAMPLE 9

Regulation of the erbB-2 Gene in Hela Cells

(Prospective Example)

The erbB-2 gene is targeted for imposed regulation. To regulate the native erbB-2 gene, a synthetic repressor protein and a transactivator protein are utilized (R. R. Beerli, D. J. Segal, B. Dreier, C. F. Barbas, III, Proc. Natl. Acad. Sci. USA 95, 14628 (1998)). This DNA-binding protein is constructed from 6 pre-defined and modular zinc finger domains (D. J. Segal, B. Dreier, R. R. Beerli, C. F. Barbas, III, Proc. Natl. Acad. Sci. USA 96, 2758 (1999)). The repressor protein contains the Kox-1 KRAB domain (J. F. Margolin et al., Proc. Natl. Acad, Sci. USA 91, 4509 (1994)), whereas the transactivator VP64 contains a tetrameric repeat of the minimal activation domain (K. Seipel, 0 Georgiev, W. Schaffner, EMBO J. 11, 4961 (1992)) derived from the herpes simplex virus protein VP16.

A derivative of the human cervical carcinoma cell line HeLa, HeLa/tet-off, is utilized (M. Gossen and H. Bujard, Proc. Natl. Acad. Sci. USA 89, 5547 (1992)). Since HeLa cells are of epithelial origin they express ErbB-2 and are well suited for studies of erbB-2 gene targeting. HeLa/tet-off cells produce the tetracycline-controlled transactivator, allowing induction of a gene of interest under the control of a tetracycline response element (TRE) by removal of tetracycline or its derivative doxycycline (Dox) from the growth medium. This system is used to place the transcription factors under chemical control. Thus, repressor and activator plasmids are constructed and subcloned into pRevTRE (Clontech) using BamHI and Clal restriction sites, and into PMX-IRES-GFP [X. Liu et al., Proc. Natl. Acad. Sci. USA 94, 10669 (1997)] using BamHI and Notl restriction sites. Fidelity of the PCR amplification are confirmed by sequencing, transfected into HeLa/tet-off cells, and 20 stable clones each are isolated and analyzed for Dox-dependent target gene regulation. The constructs are transfected into the HeLa/tet-off cell line (M. Gossen and H. Bujard, Proc. Natl. Acad. Sci. USA 89, 5547 (1992)) using Lipofectamine Plus reagent (Gibco BRL). After two weeks of selection in hygromycin-containing medium, in the presence of 2 mg/ml Dox, stable clones are isolated and analyzed for Dox-dependent regulation of ErbB-2 expression. Western blots, immunoprecipitations, Northern blots, and flow cytometric analyses are carried out essentially as described [D. Graus-Porta, R. R. Beerli, N. E. Hynes, Mol. Cell. Biol. 15, 1182 (1995)]. As a read-out of erbB-2 promoter activity, ErbB-2 protein levels are initially analyzed by Western blotting. A significant fraction of these clones will show regulation of ErbB-2 expression upon removal of Dox for 4 days, i.e., downregulation of ErbB-2 in repressor clones and upregulation in activator clones. ErbB-2 protein levels are correlated with altered levels of their specific mRNA, indicating that regulation of ErbB-2 expression is a result of repression or activation of transcription.

### EXAMPLE 10

Introduction of the Coding Regions of the E2S-KRAB, E2S-VP64, E3F-KRAB and E3F-VP64 Proteins into the Retroviral vector pM-IRES-GFP

(Prospective Example)

In order to express the E2S-KRAB, E2S-VP64, E3F-KRAB and E3F-VP64 proteins in several cell lines, their coding regions are introduced into the retroviral vector pMX-IRES-GFP.

The sequences of these constructs are selected to bind to specific regions of the ErbB-2 or ErbB-3 promoters. The coding regions are PCR amplified from pcDNA3-based expression plasmids (R. R. Beerli, D. J. Segal, B. Dreier, C. F. Barbas, III, Proc. Natl. Acad. Sci. USA 95, 14628 (1998)) and are subcloned into pRevTRE (Clontech) using BamHI and Clal restriction sites, and into pMX-IRES-GFP [X. Liu et al., Proc. Natl. Acad. Sci. USA 94, 10669 (1997)] using BamHI and NotI restriction sites. Fidelity of the PCR amplification is confirmed by sequencing. This vector expresses a single bicistronic message for the translation of the zinc finger protein and, from an internal ribosome-entry site (IRES), the green fluorescent protein (GFP). Since both coding regions share the same mRNA, their expression is physically linked to one another and GFP expression is an indicator of zinc finger expression. Virus prepared from these plasmids is then used to infect the human carcinoma cell line A431.

### EXAMPLE 11

Regulation of ErbB-2 and ErbB-3 Gene Expression

(Prospective Example)

Plasmids from Example 9 are transiently transfected into the amphotropic packaging cell line Phoenix Ampho using Lipofectamine Plus (Gibco BRL) and, two days later, culture supernatants are used for infection of target cells in the presence of 8 mg/ml polybrene. Three days after infection, cells are harvested for analysis. Three days after infection, ErbB-2 and ErbB-3 expression was measured by flow cytornetry. The results are expected to show that E2S-KRAB and E2S-VP64 compositions inhibited and enhanced ErbB-2 gene expression, respectively. The data are expected to show that E3F-KRAB and E3F-VP64 compositions inhibited and enhanced ErbB-2 gene expression, respectively.

The human erbB-2 and erbB-3 genes were chosen as model targets for the development of zinc finger-based transcriptional switches. Members of the ErbB receptor family play important roles in the development of human malignancies. In particular, erbB-2 is overexpressed as a result of gene amplification and/or transcriptional deregulation in a high percentage of human adenocarcinomas arising at numerous sites, including breast, ovary, lung, stomach, and salivary gland (Hynes, N. E. & Stem, D. F. (1994) Biochim. Biophys.Acta 1198, 165-184). Increased expression of ErbB-2 leads to constitutive activation of its intrinsic tyrosine kinase, and has been shown to cause the transformation of cultured cells. Numerous clinical studies have shown that patients bearing tumors with elevated ErbB-2 expression levels have a poorer prognosis (Hynes, N. E. & Stern, D. F. (1994) Biochim. Biophys. Acta 1198, 165-184). In addition to its involvement in human cancer, erbB-2 plays important biological roles, both in the adult and during embryonic development of mammals (Hynes, N. E. & Stem, D. F. (1994) Biochim. Biophys. Acta 1198, 165-184, Altiok, N., Bessereau, J.-L. & Changeux, J.-P. (1995) EMBO J. 14, 4258-4266, Lee, K.-F., Simon, H., Chen, H., Bates, B., Hung, M.-C. & Hauser, C. (1995) Nature 378, 394-398).

The erbB-2 promoter therefore represents an interesting test case for the development of artificial transcriptional regulators. This promoter has been characterized in detail and has been shown to be relatively complex, containing both a TATA-dependent and a TATA-independent transcriptional initiation site (Ishii, S., Imamoto, F., Yamanashi, Y., Toyoshima, K. & Yamamoto, T. (1987) Proc. Natl. Acad. Sci. USA 84, 43744378). Whereas early studies showed that polydactyl proteins could act as transcriptional regulators that specifically activate or repress transcription, these proteins bound upstream of an artificial promoter to six tandem repeats of the protein's binding site (Liu, Q., Segal, D. J., Ghiara, J. B. & Barbas, C. F. (1997) Proc. Natl. Acad. Sci. USA 94, 5525-5530). Furthermore, this study utilized polydactyl proteins that were not modified in their binding specificity Herein, we are testing the efficacy of polydactyl proteins assembled from predefined building blocks to bind a single site in the native erbB-2 and erbB-3 promoter.

For generating polydactyl proteins with desired DNA-binding specificity, the present studies have focused on the assembly of predefined zinc finger domains, which contrasts the sequential selection strategy proposed by Greisman and Pabo (Greisman, H. A. & Pabo, C. O. (1997) Science 275, 657-661). Such a strategy would require the sequential generation and selection of six zinc finger libraries for each required protein, making this experimental approach inaccessible to most laboratories and extremely time-consuming to all., Further, since it is difficult to apply specific negative selection against binding alternative sequences in this strategy, proteins may result that are relatively unspecific as was recently reported (Kim, J.-S. & Pabo, C. O. (1997) J. Biol. Chem. 272, 29795-29800).

The general utility of two different strategies for generating three-finger proteins recognizing 18 bp of DNA sequence is investigated. Each strategy was based on the modular nature of the zinc finger domain, and takes advantage of a family of zinc finger domains recognizing triplets of the 5′-(NNN)-3′. Three six-finger proteins recognizing half-sites of erbB-2 or erbB-3 target sites are generated in the first strategy by fusing the pre-defined finger 2 (F2) domain variants together using a PCR assembly strategy.

The affinity of each of the proteins for its target is determined by electrophoretic mobility-shift assays. These studies are expected to demonstrate that the zinc finger peptides have affinities comparable to Zif268 and other natural transcription factors.

The affinity of each protein for the DNA target site is determined by gel-shift analysis.

### EXAMPLE 12

Computer Modeling

(Prospective Example)

Computer models are generated using Insight II (Molecular Simulations, Inc.). Models are based on the coordinates of the co-crystal structures of Zif268-DNA (PDB accession 1AAY). The structures are not energy minimized and are presented only to suggest possible interactions. Hydrogen bonds are considered plausible when the distance between the heavy atoms was 3 (±0.3) Å and the angle formed by the heavy atoms and hydrogen is 120° C. or greater

### EXAMPLE 13

Multitarget ELISA Analysis of Zinc Finger Domains Produced by Rational Design and Site-Directed Mutagenesis

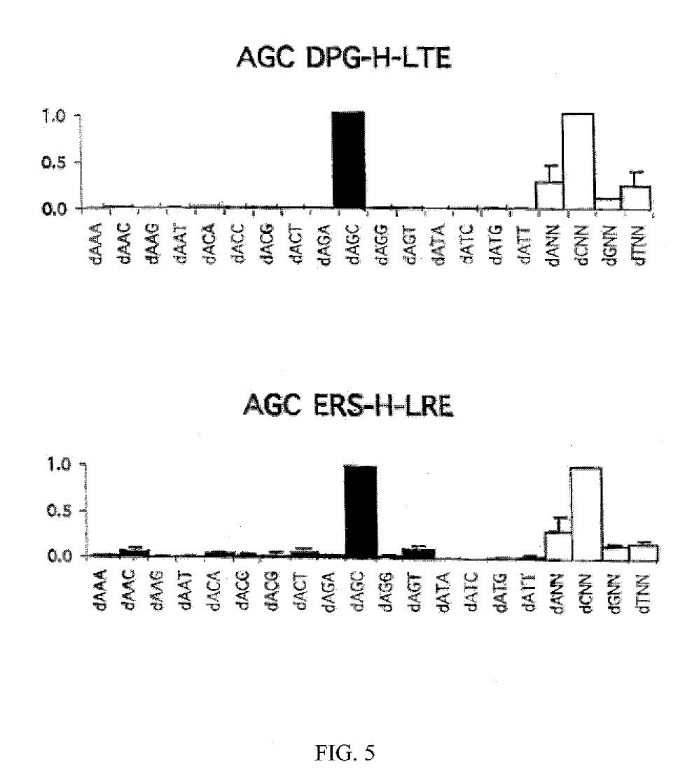

Multitarget ELISA analysis of zinc finger domains produced by rational design and site-directed mutagenesis (ERS-H-LRE (SEQ ID NO: 2) and (DPG-H-LTE (SEQ ID NO: 3)) was performed according to Example 1. The results, showing a high degree of specificity for the 5′-(ACG)-3′ subsite, are shown in FIG. 5.

TABLE 4

Summary of Protein and Nucleic Acid
Sequences Recited

| | |
|---|---|
| Heptapeptide | SEQ ID NO |

Hentapeptide Zinc Finger Moieties of the
Present Invention

| | |
|---|---|
| DPG-A-LIN | 1 |
| ERS-H-LRE | 2 |
| DPG-H-LTE | 3 |
| EPG-A-LIN | 4 |
| DRS-H-LRE | 5 |
| EPG-H-LTE | 6 |
| ERS-L-LRE | 7 |
| DRS-K-LRE | 8 |
| DPG-K-LTE | 9 |
| EPG-K-LTE | 10 |
| DPG-W-LIN | 11 |
| DPG-T-LIN | 12 |
| DPG-H-LIN | 13 |
| ERS-W-LIN | 14 |
| ERS-T-LIN | 15 |
| DPG-W-LTE | 16 |
| DPG-T-LTE | 17 |
| EPG-W-LIN | 18 |
| EPG-T-LIN | 19 |
| EPG-H-LIN | 20 |
| DRS-W-LRE | 21 |
| DRS-T-LRE | 22 |
| EPG-W-LTE | 23 |
| EPG-T-LTE | 24 |
| ERS-W-LRE | 25 |
| ERS-T-LRE | 26 |
| DPG-A-LRE | 21 |
| DPG-A-LTE | 28 |
| ERS-H-LIN | 29 |
| ERS-H-LTE | 30 |
| DPG-H-LIN | 31 |
| DPG-H-LRE | 32 |
| EPG-A-LRE | 33 |
| EPG-A-LTE | 34 |
| DRS-H-LIN | 35 |
| DRS-H-LTE | 36 |
| EPG-H-LRE | 37 |
| ERS-K-LIN | 38 |
| ERS-K-LTE | 39 |
| DRS-K-LIN | 40 |
| DRS-K-LTE | 41 |
| DPG-K-LIN | 42 |
| DPG-K-LRE | 43 |
| EPG-K-LIN | 44 |
| EPG-K-LRE | 45 |
| DPG-W-LRE | 46 |
| DPG-T-LRE | 47 |
| DPG-H-LRE | 48 |
| DPG-H-LTE | 49 |
| ERS-W-LTE | 50 |
| ERS-T-LTE | 51 |
| EPG-W-LRE | 52 |
| EPG-T-LRE | 53 |
| DRS-W-LIN | 54 |
| DRS-W-LTE | 55 |
| DRS-T-LIN | 56 |
| DRS-T-LTE | 57 |

Other Heptapeptide Zinc Finger Moieties Recited

| | |
|---|---|
| RSD-E-LKR | 58 |
| SPA-D-LTN | 59 |
| HIS-N-FCR | 60 |
| RED-N-LHT | 61 |
| RSD-H-LTT | 62 |
| DAS-H-LHT | 63 |
| ERS-K-LAR | 64 |
| DPG-H-LVR | 55 |
| DPG-A-LVR | 66 |
| ERS-K-LRA | 67 |
| DPG-H-LRV | 68 |
| DPG-A-LRV | 69 |
| DPG-S-LRV | 70 |
| RSD-H-LTN | 71 |
| RSD-H-LAE | 72 |
| RSD-N-LKN | 73 |
| RSD-T-LSN | 74 |
| RTD-T-LRD | 75 |
| RRD-A-LNV | 76 |
| SRD-A-LNV | 77 |
| RSD-T-LRD | 78 |
| HRT-T-LLN | 79 |
| VKD-Y-LTK | 80 |
| KNW-K-LQA | 81 |
| HIS-N-FCR | 82 |
| AQY-M-LVV | 83 |
| QST-N-LKS | 84 |
| LDF-N-LRT | 85 |
| RSD-H-LTT | 86 |
| RKD-N-MTA | 87 |
| QSS-N-LIT | 88 |
| QRS-A-LTV | 89 |
| QRA-N-LRA | 90 |
| QSG-S-LTR | 91 |
| DSG-N-LRV | 92 |
| TSH-G-LTT | 93 |
| HRT-T-LTN | 94 |
| SPA-D-LTR | 95 |
| SHS-D-LVR | 96 |
| HIS-N-FCR | 97 |
| HKN-A-LQN | 98 |
| HRT-T-LLN | 99 |

Other Protein or Peptide Sequences

| | |
|---|---|
| Protein or Peptide Sequence | SEQ ID NO |

| | |
|---|---|
| TGEKP (Linker) | 100 |
| TGGGGSGGGGTGEKP (Linker) | 101 |
| LRQKDGGGSERP (Linker) | 102 |
| LRQKDGERP (Linker) | 103 |
| GGRGRGRGRQ (Linker) | 104 |
| QNKKGGSGDGKKKQHI (Linker) | 105 |
| TGGERP (Linker) | 106 |
| ATGEKP (Linker) | 107 |
| DALDDFDLDML (Activation domain) | 108 |
| GGGSGGGGEGP (Linker) | 116 |

Nucleotide Sequences

| | |
|---|---|
| Nucleotide Sequence | SEQ ID NO |

| | |
|---|---|
| GATCNNGCG | 109 |
| GATANNGCG | 110 |
| GAGGAAGTTTGCCACCAGTGGCAACCTGGTGAGGCATACCA | 111 |
| AAATC | |
| GTAAAACGACGGCCAGTGCCAAGC | 112 |
| GGCCGCN′N′N′ATCGAGTTTTCTCGATNNNGCGGCC | 113 |
| GATANNGCG | 114 |
| GCGNNNGCG | 115 |

### ADVANTAGES OF THE INVENTION

The present invention provides versatile binding proteins for nucleic acid sequences, particularly DNA sequences. These binding proteins can be coupled with transcription modulators and can therefore be utilized for the upregulation or downregulation of particular genes in a specific manner. These binding proteins can, therefore, be used in gene therapy or protein therapy for the treatment of cancer, autoimmune diseases, metabolic disorders, developmental disorders, and other diseases or conditions associated with the dysregulation of gene expression.

The polypeptides, polypeptide compositions, isolated heptapeptides, pharmaceutical compositions, and methods according to the present invention possess industrial applicability for the preparation of medicaments that can treat diseases and conditions treatable by the control or modulation of gene expression.

With respect to ranges of values, the invention encompasses each intervening value between the upper and lower limits of the range to-at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a.,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled Those skilled in the art will recognize, or will be able to ascertain using no more than routine experimentation, many equivalents to the specific embodiments of the invention described. Such equivalents are intended to be encompassed by the following claims.

We claim:

1. An isolated and purified zinc finger nucleotide binding polypeptide comprising a nucleotide binding region of from 5 to 10 amino acid residues, which region binds preferentially to a target nucleotide of the formula AGC, where N is A, C, G or T.

2. The polypeptide of claim 1 wherein the binding region competes for binding with a polypeptide that includes therein any of SEQ ID NO: 1 through SEQ ID NO: 57.

3. The polypeptide of claim 2 wherein the binding region competes for binding with a polypeptide that includes therein any of SEQ ID NO: 1 through SEQ ID NO: 10.

4. The polypeptide of claim 3 wherein the binding region competes for binding with a polypeptide that includes therein any of SEQ ID NO: 1 through SEQ ID NO: 3.

5. The polypeptide of claim 1 wherein the binding region has the amino acid sequence of any of SEQ ID NO: 1 through SEQ ID NO: 57.

6. The polypeptide of claim 5 wherein the binding region has the amino acid sequence of any of SEQ ID NO: 1 through SEQ ID NO: 10.

7. The polypeptide of claim 6 wherein the binding region has the amino acid sequence of any of SEQ ID NO: 1 through SEQ ID NO: 3.

8. The polypeptide of claim 1 wherein the nucleotide binding region is 7 residues and has α-helical structure.

9. The polypeptide of claim 1 wherein the binding region has an amino acid sequence selected from the group consisting of:
(a) the binding region of the amino acid sequence of any of SEQ ID NO: 1 through SEQ ID NO: 57; and
(b) a binding region differing from the amino acid sequence of any of SEQ ID NO: 1 through SEQ ID NO: 57 by no more than two conservative amino acid substitutions, wherein the dissociation constant is no greater than 125% of that of the polypeptide before the substitutions are made, and wherein a conservative amino acid substitution is one of the following substitutions: Ala/Gly or Ser; Arg/Lys; Asn/Gln or His; Asp/Glu; Cys/Ser, Gln/Asn; Gly/Asp; Gly/Ala or Pro; His/Asn or Gln; Ile/Leu or Val; Leu/ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu.

10. The polypeptide of claim 9 wherein the binding region differs from the amino acid sequence of any of SEQ ID NO: 1 through SEQ ID NO: 10 by no more than two conservative amino acid substitutions.

11. The polypeptide of claim 10 wherein the binding region differs from the amino acid sequence of any of SEQ ID NO: 1 through SEQ ID NO. 3 by no more than two conservative amino acid substitutions.

12. The polypeptide of claim 1, wherein the nucleotide binding region comprises a 7-amino acid zinc finger domain in which the seven amino acids of the domain are: numbered from −1 to 6, and wherein the domain is selected from the group consisting of:
(a) a zinc finger nucleotide binding domain specifically binding the nucleotide sequence 5′-(AGC)-3′, wherein the amino acid residue of the domain numbered −1 is selected from the group consisting of Q, N, S, G, H, and D;
(b) a zinc finger nucleotide binding domain specifically binding the nucleotide sequence 5′-(AGC)-3′, wherein the amino acid residue of the domain numbered 3 is selected from the group consisting of W, T, and H;
(c) a zinc finger nucleotide binding domain specifically binding the nucleotide sequence 5′-(AGC)-3′ wherein the amino acid residue of the domain numbered 4 is selected from the group consisting of L, V, I, and C;
(d) a zinc finger nucleotide binding domain specifically binding the nucleotide sequence 5′-(AGC)-3′ wherein the amino acid residue of the domain numbered 6 is selected from the group consisting of A, R, N, D, Q, E, T, and V; and
(e) a zinc finger nucleotide binding domain specifically binding the nucleotide sequence 5′-(AGC)-3′ wherein the amino acid residue of the domain numbered −1 is selected from the group consisting of D and E and wherein the residues of the domain numbering 4 through 6 are selected from the group consisting of LIN, LRE, and LTE.

13. A polypeptide composition comprising a plurality of the polypeptides of claim 1, wherein the polypeptides are operatively linked to each other.

14. The polypeptide composition of claim 13 wherein the polypeptides are operatively linked via a flexible peptide linker of from 5 to 15 amino acid residues.

15. The polypeptide composition of claim 14 wherein the linker has a sequence selected from the group consisting of SEQ ID NO: 100 through SEQ ID NO: 107 and SEQ ID NO: 116.

16. The polypeptide composition of claim 13 wherein the composition comprises from 2 to 18 polypeptides.

17. The polypeptide composition of claim 16 wherein the composition comprises from 2 to 12 polypeptides.

18. The polypeptide composition of claim 17 wherein the composition comprises from 2 to 6 polypeptides.

19. The polypeptide composition of claim 13 wherein the composition further comprises at least one polypeptide with a binding region that binds a nucleotide subsite of the sequence 5′-(ANN)-3′, 5′-(CNN)-3′, 5′-(GNN)-3′, or 5′-(TNN)-3′.

20. The polypeptide composition of claim 13 wherein the binding region of each polypeptide competes for binding with a polypeptide that includes therein any of SEQ ID NO: 1 through SEQ ID NO: 57.

21. The polypeptide composition of claim 20 wherein the binding region of each polypeptide competes for binding with a polypeptide that includes therein any of SEQ ID NO: 1 through SEQ ID NO: 10.

22. The polypeptide composition of claim 21 wherein the binding region of each polypeptide competes for binding with a polypeptide that includes therein any of SEQ ID NO: 1 through SEQ ID NO: 3.

23. The polypeptide composition of claim 13 wherein the binding region of each polypeptide has the amino acid sequence of any of SEQ ID NO: 1 through SEQ ID NO: 57.

24. The polypeptide composition of claim 23 wherein the binding region of each polypeptide has the amino acid sequence of any of SEQ ID NO: 1 through SEQ ID NO: 10.

25. The polypeptide composition of claim 24 wherein the binding region of each polypeptide has the amino acid sequence of any of SEQ ID NO: 1 through SEQ ID NO: 3.

26. The polypeptide composition of claim 13 wherein the nucleotide binding region of each polypeptide is 7 residues and has α-helical structure.

27. The polypeptide composition of claim 13 wherein the binding region of each polypeptide has an amino acid sequence selected from the group consisting of:
(a) the binding region of the amino acid sequence of any of SEQ ID NO: 1 through SEQ ID NO: 57; and
(b) a binding region differing from the amino acid sequence of any of SEQ ID NO: 1 through SEQ ID NO: 57 by no more than two conservative amino acid substitutions, wherein the dissociation constant is no greater than 125% of that of the polypeptide before the substitutions are made, and wherein a conservative amino acid substitution is one of the following substitutions: Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu.

28. The polypeptide composition of claim 27 wherein the binding region of each polypeptide differs from the amino acid sequence of any of SEQ ID NO: 1 through SEQ ID NO: 10 by no more than two conservative amino acid substitutions.

29. The polypeptide composition of claim 28 wherein the binding region of each polypeptide differs from the amino acid sequence of any of SEQ ID NO: 1 through SEQ ID NO: 3 by no more than two conservative amino acid substitutions.

30. The polypeptide composition of claim 13, wherein the nucleotide binding region of each polypeptide comprises a 7-amino acid zinc finger domain in which the seven amino acids of the domain are numbered from −1 to 6, and wherein the domain is selected from the group consisting of:
(a) a zinc finger nucleotide binding domain specifically binding the nucleotide sequence 5′-(AGC)-3′, wherein the amino acid residue of the domain numbered −1 is selected from the group consisting of Q, N, S, G, H, and D;
(b) a zinc finger nucleotide binding domain specifically binding the nucleotide sequence 5′-(AGC)-3′, wherein the amino acid residue of the domain numbered 3 is selected from the group consisting of W, T, and H;
(c) a zinc finger nucleotide binding domain specifically binding the nucleotide sequence 5′-(AGC)-3′ wherein the amino acid residue of the domain numbered 4 is selected from the group consisting of L, V, I, and C; and
(d) a zinc finger nucleotide binding domain specifically binding the nucleotide sequence 5′-(AGC)-3′ wherein the amino acid residue of the domain numbered 6 is selected from the group consisting of A, R, N, D, Q, E, T, and V, and (e) a zinc finger nucleotide binding domain specifically binding the nucleotide sequence 5′-(AGC)-3′ wherein the amino acid residue of the domain numbered −1 is selected from the group consisting of D and E and wherein the residues of the domain numbering 4 through 6 are selected from the group consisting of LIN, LRE, and LTE.

31. The polypeptide composition of claim 13 wherein the polypeptide composition comprises a bispecific zinc finger protein comprising two halves, each half comprising six zinc finger nucleotide binding domains, where at least one of the halves includes at least one domain binding a target nucleotide sequence of the form 5′-(AGC)-3′, such that the two halves of the bispecific zinc fingers can operate independently.

32. The polypeptide composition of claim 31 wherein the two halves of the bispecific zinc finger protein are joined by a linker.

33. The polypeptide composition of claim 13 wherein the polypeptide composition further comprises the nuclease catalytic domain of Fokl such that the polypeptide composition directs site-specific cleavage at a chosen genomic target.

34. An isolated heptapeptide having an α-helical structure and that binds preferentially to a target nucleotide of the formula AGC.

35. The isolated heptapeptide of claim 34 wherein the heptapeptide has the amino acid sequence of any of SEQ ID NO: 1 through SEQ ID NO: 57.

36. The isolated heptapeptide of claim 35 wherein the heptapeptide has the amino acid sequence of any of SEQ ID NO: 1 through SEQ ID NO: 10.

37. The isolated heptapeptide of claim 36 wherein the heptapeptide has the amino acid sequence of any of SEQ ID NO: 1 through SEQ ID NO: 3.

38. The isolated heptapeptide of claim 34 wherein the heptapeptide competes for binding with a polypeptide that includes therein any of SEQ ID NO: 1 through SEQ ID NO: 57.

39. The isolated heptapeptide of claim 38 wherein the heptapeptide competes for binding with a polypeptide that includes therein any of SEQ ID NO: 1 through SEQ ID NO: 10.

40. The isolated heptapeptide of claim 39 wherein the heptapeptide competes for binding with a polypeptide that includes therein any of SEQ ID NO: 1 through SEQ ID NO: 3.

41. The isolated heptapeptide of claim 34 wherein the heptapeptide has an amino acid sequence selected from the group consisting of:
(a) the binding region of the amino acid sequence of any of SEQ ID NO: 1 through SEQ ID NO: 57; and
(b) a binding region differing from the amino acid sequence of any of SEQ ID NO: 1 through SEQ ID NO: 57 by no more than two conservative amino acid substitutions, wherein the dissociation constant is no greater than 125% of that of the heptapeptide before the substitutions are made, and wherein a conservative amino acid substitution is one of the following substitutions: Ala/Gly or Ser; Arg/Lys; Asn/Gln or His; Asp/Glu; Cys/Ser; Gln/Asn; Gly/Asp, Gly/Ala or Pro; His/Asn or Gln; Ile/Leu or Val; Leulile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu.

42. The isolated heptapeptide of claim 41 wherein the binding region differs from the amino acid sequence of any of SEQ ID NO: 1 through SEQ ID NO: 10 by no more than two conservative amino acid substitutions.

43. The isolated heptapeptide of claim 42 wherein the binding region differs from the amino acid sequence of any of SEQ ID NO: 1 through SEQ ID NO: 3 by no more than two conservative amino acid substitutions.

44. The isolated heptapeptide of claim 34 wherein the heptapeptide is selected from the group consisting of:
(a) an isolated heptapeptide specifically binding the nucleotide sequence 5′-(AGC)-3′, wherein the amino acid residue of the domain numbered −1 is selected from the group consisting of Q, N, S, G, H, and D,
(b) an isolated heptapeptide specifically binding the nucleotide sequence 5′-(AGC)-3′, wherein the amino acid residue of the domain numbered 3 is selected from the group consisting of W, T, and H;
(c) an isolated heptapeptide specifically binding the nucleotide sequence 51-(AGC)-3′ wherein the amino acid residue of the domain numbered 4 is selected from the group consisting of L, V, I, and C;
(d) an isolated heptapeptide specifically binding the nucleotide sequence 5′-(AGC)-3′ wherein the amino acid residue of the domain numbered 6 is selected from the group consisting of A, R, N, D, Q, E, T, and V; and
(e) a zinc finger nucleotide binding domain specifically binding the nucleotide sequence 5′-(AGC)-3′ wherein the amino acid residue of the domain numbered −1 is selected from the group consisting of D and E and wherein the residues of the domain numbering 4 through 6 are selected from the group consisting of LIN, LRE, and LTE.

45. The polypeptide of claim 1 operatively linked to one or more transcription regulating factors.

46. The polypeptide of claim 45 wherein the transcription regulating factor is a repressor of transcription.

47. The polypeptide of claim 45 wherein the transcription regulating factor is an activator of transcription.

48. The polypeptide of claim 45 wherein the transcription regulating factor is selected from the group consisting of histone deacetylase and a modulator of histone deacetylase expression.

49. The polypeptide composition of claim 13 operatively linked to one or more transcription regulating factors.

50. The polypeptide composition of claim 49 wherein the transcription regulating factor is a repressor of transcription.

51. The polypeptide composition of claim 49 wherein the transcription regulating factor is an activator of transcription.

52. The polypeptide composition of claim 49 wherein the transcription regulating factor is selected from the group consisting of histone deacetylase and a modulator of histone deacetylase expression.

53. An isolated and purified polynucleotide that encodes the polypeptide of claim 1.

54. An isolated and purified polynucleotide that encodes the polypeptide composition of claim 13.

55. An isolated and purified polynucleotide that encodes the isolated heptapeptide of claim 34.

56. A vector comprising the isolated and purified polynucleotide of claim 53.

57. A vector comprising the isolated and purified polynucleotide of claim 54.

58. A vector comprising the isolated and purified polynucleotide of claim 55.

59. A host cell transformed or transfected with the vector of claim 56.

60. A host cell transformed or transfected with the vector of claim 57.

61. A host cell transformed or transfected with the vector of claim 58.

62. A host cell transformed or transfected with the polynucleotide of claim 53.

63. A host cell transformed or transfected with the polynucleotide of claim 54.

64. A host cell transformed or transfected with the polynucleotide of claim 55.

65. An isolated and purified polynucleotide selected from the group consisting of:
(a) an isolated and purified polynucleotide that encodes the polypeptide of claim 1; and
(b) nucleic acid sequences that are at least 95% identical with the sequences of (a), provided that the nucleic acid sequences are translated into polypeptides that possess the activity of the polypeptide of claim 1, including specific nucleic acid binding activity.

66. An isolated and purified polynucleotide selected from the group consisting of:
(a) an isolated and purified polynucleotide that encodes the polypeptide composition of claim 13; and
(b) nucleic acid sequences that are at least 95% identical with the sequences of (a), provided that the nucleic acid sequences are translated into polypeptides that possess the activity of the polypeptide composition of claim 20, including specific nucleic acid binding activity.

67. An isolated and purified polynucleotide selected from the group consisting of:
(a) an isolated and purified polynucleotide that encodes the heptapeptide of claim 34; and
(b) nucleic acid sequences that are at least 95% identical with the sequences of (a), provided that the nucleic acid sequences are translated into polypeptides that possess the activity of the heptapeptide of claim 51, including specific nucleic acid binding activity.

68. A process of regulating expression of a nucleotide sequence that contains the sequence 5′-(AGC)ₙ-3′, where n is 2 to 12, the process comprising exposing the nucleotide sequence to an effective amount of the polypeptide composition of claim 13.

69. The process of claim 68 wherein the sequence 5′-(AGC)ₙ-3′ is located in the transcribed region of the nucleotide sequence.

70. The process of claim 68 wherein the sequence 5′-(AGC)ₙ-3′ is located in a promoter region of the nucleotide sequence.

71. The process of claim 68 wherein the sequence 5′-(AGC)ₙ-3′ is located within an expressed sequence tag.

72. The process of claim 68 wherein the polypeptide composition is operatively linked to one or more transcription regulating factors.

73. The process of claim 68 wherein the nucleotide sequence is a gene.

74. A pharmaceutical composition comprising:
(a) a therapeutically effective amount of the polypeptide of claim 1; and
(b) a pharmaceutically acceptable carrier.

75. A pharmaceutical composition comprising:
(a) a therapeutically effective amount of the polypeptide composition of claim 13; and
(b) a pharmaceutically acceptable carrier.

76. A pharmaceutical composition comprising:
(a) a therapeutically effective amount of the heptapeptide of claim 34; and
(b) a pharmaceutically acceptable carrier.

77. A pharmaceutical composition comprising:
(a) a therapeutically effective amount of the polynucleotide of claim 53; and
(b) a pharmaceutically acceptable carrier.

78. A pharmaceutical composition comprising:
(a) a therapeutically effective amount of the polynucleotide of claim 54; and
(b) a pharmaceutically acceptable carrier.

79. A pharmaceutical composition comprising:
(a) a therapeutically effective amount of the polynucleotide of claim 55; and
(b) a pharmaceutically acceptable carrier.

80. A pharmaceutical composition comprising:
(a) a therapeutically effective amount of the polynucleotide of claim 65; and
(b) a pharmaceutically acceptable carrier.

81. A pharmaceutical composition comprising:
(a) a therapeutically effective amount of the polynucleotide of claim 66; and
(b) a pharmaceutically acceptable carrier.

82. A pharmaceutical composition comprising:
(a) a therapeutically effective amount of the polynucleotide of claim 67; and
(b) a pharmaceutically acceptable carrier.


SEQUENCE LISTING (116 sequences)

SEQ ID NO: 1 - protein, 7 aa, Artificial
  Synthetically Generated Peptide
DPGALIN

SEQ ID NO: 2 - protein, 7 aa, Artificial
  Synthetically Generated Peptide
ERSHLRE

SEQ ID NO: 3 - protein, 7 aa, Artificial
  Synthetically Generated Peptide
DPGHLTE

SEQ ID NO: 4 - protein, 7 aa, Artificial
  Synthetically Generated Peptide
EPGALIN

SEQ ID NO: 5 - protein, 7 aa, Artificial
  Synthetically Generated Peptide
DRSHLRE

SEQ ID NO: 6 - protein, 7 aa, Artificial
  Synthetically Generated Peptide
EPGHLTE

SEQ ID NO: 7 - protein, 7 aa, Artificial
  Synthetically Generated Peptide
ERSLLRE

SEQ ID NO: 8 - protein, 7 aa, Artificial
  Synthetically Generated Peptide
DRSKLRE

SEQ ID NO: 9 - protein, 7 aa, Artificial
  Synthetically Generated Peptide
DPGKLTE

SEQ ID NO: 10 - protein, 7 aa, Artificial
  Synthetically Generated Peptide
EPGKLTE

SEQ ID NO: 11 - protein, 7 aa, Artificial
  Synthetically Generated Peptide
DPGWLIN

SEQ ID NO: 12 - protein, 7 aa, Artificial
  Synthetically Generated Peptide
DPGTLIN

SEQ ID NO: 13 - protein, 7 aa, Artificial
  Synthetically Generated Peptide
DPGHLIN

SEQ ID NO: 14 - protein, 7 aa, Artificial
  Synthetically Generated Peptide
ERSWLIN

SEQ ID NO: 15 - protein, 7 aa, Artificial
  Synthetically Generated Peptide
ERSTLIN

SEQ ID NO: 16 - protein, 7 aa, Artificial
  Synthetically Generated Peptide
DPGWLTE

SEQ ID NO: 17 - protein, 7 aa, Artificial
  Synthetically Generated Peptide
DPGTLTE

SEQ ID NO: 18 - protein, 7 aa, Artificial
  Synthetically Generated Peptide
EPGWLIN

SEQ ID NO: 19 - protein, 7 aa, Artificial
  Synthetically Generated Peptide
EPGTLIN

SEQ ID NO: 20 - protein, 7 aa, Artificial
  Synthetically Generated Peptide
EPGHLIN

SEQ ID NO: 21 - protein, 7 aa, Artificial
  Synthetically Generated Peptide
DRSWLRE

SEQ ID NO: 22 - protein, 7 aa, Artificial
  Synthetically Generated Peptide
DRSTLRE

SEQ ID NO: 23 - protein, 7 aa, Artificial
  Synthetically Generated Peptide
EPGWLTE

SEQ ID NO: 24 - protein, 7 aa, Artificial
  Synthetically Generated Peptide
EPGTLTE

SEQ ID NO: 25 - protein, 7 aa, Artificial
  Synthetically Generated Peptide
ERSWLRE

SEQ ID NO: 26 - protein, 7 aa, Artificial
  Synthetically Generated Peptide
ERSTLRE

SEQ ID NO: 27 - protein, 7 aa, Artificial
  Synthetically Generated Peptide
DPGALRE

SEQ ID NO: 28 - protein, 7 aa, Artificial
  Synthetically Generated Peptide
DPGALTE

SEQ ID NO: 29 - protein, 7 aa, Artificial
  Synthetically Generated Peptide
ERSHLIN

SEQ ID NO: 30 - protein, 7 aa, Artificial
  Synthetically Generated Peptide
ERSHLTE

SEQ ID NO: 31 - protein, 7 aa, Artificial
  Synthetically Generated Peptide
DPGHLIN

SEQ ID NO: 32 - protein, 7 aa, Artificial
  Synthetically Generated Peptide
DPGHLRE

SEQ ID NO: 33 - protein, 7 aa, Artificial
  Synthetically Generated Peptide
EPGALRE

SEQ ID NO: 34 - protein, 7 aa, Artificial
  Synthetically Generated Peptide
EPGALTE

SEQ ID NO: 35 - protein, 7 aa, Artificial
  Synthetically Generated Peptide
DRSHLIN

SEQ ID NO: 36 - protein, 7 aa, Artificial
  Synthetically Generated Peptide
DRSHLTE

SEQ ID NO: 37 - protein, 7 aa, Artificial
  Synthetically Generated Peptide
EPGHLRE

SEQ ID NO: 38 - protein, 7 aa, Artificial
  Synthetically Generated Peptide
ERSKLIN

SEQ ID NO: 39 - protein, 7 aa, Artificial
  Synthetically Generated Peptide
ERSKLTE

SEQ ID NO: 40 - protein, 7 aa, Artificial
  Synthetically Generated Peptide
DRSKLIN

SEQ ID NO: 41 - protein, 7 aa, Artificial
  Synthetically Generated Peptide
DRSKLTE

SEQ ID NO: 42 - protein, 7 aa, Artificial
  Synthetically Generated Peptide
DPGKLIN

SEQ ID NO: 43 - protein, 7 aa, Artificial
  Synthetically Generated Peptide
DPGKLRE

SEQ ID NO: 44 - protein, 7 aa, Artificial
  Synthetically Generated Peptide
EPGKLIN

SEQ ID NO: 45 - protein, 7 aa, Artificial
  Synthetically Generated Peptide
EPGKLRE

SEQ ID NO: 46 - protein, 7 aa, Artificial
  Synthetically Generated Peptide
DPGWLRE

SEQ ID NO: 47 - protein, 7 aa, Artificial
  Synthetically Generated Peptide
DPGTLRE

SEQ ID NO: 48 - protein, 7 aa, Artificial
  Synthetically Generated Peptide
DPGHLRE

SEQ ID NO: 49 - protein, 7 aa, Artificial
  Synthetically Generated Peptide
DPGHLTE

SEQ ID NO: 50 - protein, 7 aa, Artificial
  Synthetically Generated Peptide
ERSWLTE

SEQ ID NO: 51 - protein, 7 aa, Artificial
  Synthetically Generated Peptide
ERSTLTE

SEQ ID NO: 52 - protein, 7 aa, Artificial
  Synthetically Generated Peptide
EPGWLRE

SEQ ID NO: 53 - protein, 7 aa, Artificial
  Synthetically Generated Peptide
EPGTLRE

SEQ ID NO: 54 - protein, 7 aa, Artificial
  Synthetically Generated Peptide
DRSWLIN

SEQ ID NO: 55 - protein, 7 aa, Artificial
  Synthetically Generated Peptide
DRSWLTE

SEQ ID NO: 56 - protein, 7 aa, Artificial
  Synthetically Generated Peptide
DRSTLIN

SEQ ID NO: 57 - protein, 7 aa, Artificial
  Synthetically Generated Peptide
DRSTLTE

SEQ ID NO: 58 - protein, 7 aa, Artificial
  Synthetically Generated Peptide
RSDELKR

SEQ ID NO: 59 - protein, 7 aa, Artificial
  Synthetically Generated Peptide
SPADLTN

SEQ ID NO: 60 - protein, 7 aa, Artificial
  Synthetically Generated Peptide
HISNFCR

SEQ ID NO: 61 - protein, 7 aa, Artificial
  Synthetically Generated Peptide
REDNLHT

SEQ ID NO: 62 - protein, 7 aa, Artificial
  Synthetically Generated Peptide
RSDHLTT

SEQ ID NO: 63 - protein, 7 aa, Artificial
  Synthetically Generated Peptide
DASHLHT

SEQ ID NO: 64 - protein, 7 aa, Artificial
  Synthetically Generated Peptide
ERSKLAR

SEQ ID NO: 65 - protein, 7 aa, Artificial
  Synthetically Generated Peptide
DPGHLVR

SEQ ID NO: 66 - protein, 7 aa, Artificial
  Synthetically Generated Peptide
DPGALVR

SEQ ID NO: 67 - protein, 7 aa, Artificial
  Synthetically Generated Peptide
ERSKLRA

SEQ ID NO: 68 - protein, 7 aa, Artificial
  Synthetically Generated Peptide
DPGHLRV

SEQ ID NO: 69 - protein, 7 aa, Artificial
  Synthetically Generated Peptide
DPGALRV

SEQ ID NO: 70 - protein, 7 aa, Artificial
  Synthetically Generated Peptide
DPGSLRV

SEQ ID NO: 71 - protein, 7 aa, Artificial
  Synthetically Generated Peptide
RSDHLTN

SEQ ID NO: 72 - protein, 7 aa, Artificial
  Synthetically Generated Peptide
RSDHLAE

SEQ ID NO: 73 - protein, 7 aa, Artificial
  Synthetically Generated Peptide
RSDNLKN

SEQ ID NO: 74 - protein, 7 aa, Artificial
  Synthetically Generated Peptide
RSDTLSN

SEQ ID NO: 75 - protein, 7 aa, Artificial
  Synthetically Generated Peptide
RTDTLRD

SEQ ID NO: 76 - protein, 7 aa, Artificial
  Synthetically Generated Peptide
RRDALNV

SEQ ID NO: 77 - protein, 7 aa, Artificial
  Synthetically Generated Peptide
SRDALNV

SEQ ID NO: 78 - protein, 7 aa, Artificial
  Synthetically Generated Peptide
RSDTLRD

SEQ ID NO: 79 - protein, 7 aa, Artificial
  Synthetically Generated Peptide
HRTTLLN

SEQ ID NO: 80 - protein, 7 aa, Artificial
  Synthetically Generated Peptide
VKDYLTK

SEQ ID NO: 81 - protein, 7 aa, Artificial
  Synthetically Generated Peptide
KNWKLQA

SEQ ID NO: 82 - protein, 7 aa, Artificial
  Synthetically Generated Peptide
HISNFCR

SEQ ID NO: 83 - protein, 7 aa, Artificial
  Synthetically Generated Peptide
AQYMLVV

SEQ ID NO: 84 - protein, 7 aa, Artificial
  Synthetically Generated Peptide
QSTNLKS

SEQ ID NO: 85 - protein, 7 aa, Artificial
  Synthetically Generated Peptide
LDFNLRT

SEQ ID NO: 86 - protein, 7 aa, Artificial
  Synthetically Generated Peptide
RSDHLTT

SEQ ID NO: 87 - protein, 7 aa, Artificial
  Synthetically Generated Peptide
RKDNMTA

SEQ ID NO: 88 - protein, 7 aa, Artificial
  Synthetically Generated Peptide
QSSNLIT

SEQ ID NO: 89 - protein, 7 aa, Artificial
  Synthetically Generated Peptide
QRSALTV

SEQ ID NO: 90 - protein, 7 aa, Artificial
  Synthetically Generated Peptide
QRANLRA

SEQ ID NO: 91 - protein, 7 aa, Artificial
  Synthetically Generated Peptide
QSGSLTR

SEQ ID NO: 92 - protein, 7 aa, Artificial
  Synthetically Generated Peptide
DSGNLRV

SEQ ID NO: 93 - protein, 7 aa, Artificial
  Synthetically Generated Peptide
TSHGLTT

SEQ ID NO: 94 - protein, 7 aa, Artificial
  Synthetically Generated Peptide
HRTTLTN

SEQ ID NO: 95 - protein, 7 aa, Artificial
  Synthetically Generated Peptide
SPADLTR

SEQ ID NO: 96 - protein, 7 aa, Artificial
  Synthetically Generated Peptide
SHSDLVR

SEQ ID NO: 97 - protein, 7 aa, Artificial
  Synthetically Generated Peptide
HISNFCR

SEQ ID NO: 98 - protein, 7 aa, Artificial
  Synthetically Generated Peptide
HKNALQN

SEQ ID NO: 99 - protein, 7 aa, Artificial
  Synthetically Generated Peptide
HRTTLLN

SEQ ID NO: 100 - protein, 5 aa, Artificial
  Synthetically Generated Peptide
TGEKP

SEQ ID NO: 101 - protein, 15 aa, Artificial
  Synthetically Generated Peptide
TGGGGSGGGGTGEKP

SEQ ID NO: 102 - protein, 12 aa, Artificial
  Synthetically Generated Peptide
LRQKDGGGSERP

SEQ ID NO: 103 - protein, 9 aa, Artificial
  Synthetically Generated Peptide
LRQKDGERP

SEQ ID NO: 104 - protein, 10 aa, Artificial
  Synthetically Generated Peptide
GGRGRGRGRQ

SEQ ID NO: 105 - protein, 16 aa, Artificial
  Synthetically Generated Peptide
QNKKGGSGDGKKKQHI

SEQ ID NO: 106 - protein, 6 aa, Artificial
  Synthetically Generated Peptide
TGGERP

SEQ ID NO: 107 - protein, 6 aa, Artificial
  Synthetically Generated Peptide
ATGEKP

SEQ ID NO: 108 - protein, 11 aa, Artificial
  Synthetically Generated Peptide
DALDDFDLDML

SEQ ID NO: 109 - DNA, 9 bp, Artificial
  Synthetically Generated Oligonucleotide
  misc_feature (5)..(6): n is a, c, g, or t
gatcnngcg

SEQ ID NO: 110 - DNA, 9 bp, Artificial
  Synthetically Generated Oligonucleotide
  misc_feature (5)..(6): n is a, c, g, or t
gatanngcg

SEQ ID NO: 111 - DNA, 46 bp, Artificial
  Synthetically Generated Oligonucleotide
gaggaagtttgccaccagtggcaacctggtgaggcataccaaaatc

SEQ ID NO: 112 - DNA, 24 bp, Artificial
  Synthetically Generated Oligonucleotide
gtaaaacgacggccagtgccaagc

SEQ ID NO: 113 - DNA, 34 bp, Artificial
  Synthetically Generated Oligonucleotide
  misc_feature (7)..(9): n is a, c, g, or t
  misc_feature (26)..(28): n is a, c, g, or t
ggccgcnnnatcgagttttctcgatnnngcggcc

SEQ ID NO: 114 - DNA, 9 bp, Artificial
  Synthetically Generated Oligonucleotide
  misc_feature (5)..(6): n is a, c, g, or t
gatanngcg

SEQ ID NO: 115 - DNA, 9 bp, Artificial
  Synthetically Generated Oligonucleotide
  misc_feature (4)..(6): n is a, c, g, or t
gcgnnngcg

SEQ ID NO: 116 - protein, 11 aa, Artificial
  Synthetically Generated Peptide
GGGSGGGGEGP